(19) United States
(12) Patent Application Publication
(10) Pub. No.: US 2014/0030711 A1
(43) Pub. Date: Jan. 30, 2014

(54) METHODS AND BIOMARKERS FOR DETECTION OF LYMPHOMA

(71) Applicants: Kojo S.J. Elenitoba-Johnson, Ann Arbor, MI (US); Megan S. Lim, Ann Arbor, MI (US); Mark J. Kiel, Ann Arbor, MI (US); Thirunavukkarasu Velusamy, Ann Arbor, MI (US)

(72) Inventors: Kojo S.J. Elenitoba-Johnson, Ann Arbor, MI (US); Megan S. Lim, Ann Arbor, MI (US); Mark J. Kiel, Ann Arbor, MI (US); Thirunavukkarasu Velusamy, Ann Arbor, MI (US)

(73) Assignee: THE REGENTS OF THE UNIVERSITY OF MICHIGAN, Ann Arbor, MI (US)

(21) Appl. No.: 13/931,177

(22) Filed: Jun. 28, 2013

Related U.S. Application Data
(60) Provisional application No. 61/666,445, filed on Jun. 29, 2012.

(51) Int. Cl. C12Q 1/68

(52) U.S. Cl. CPC C12Q 1/6886
USPC 435/6.11

(57) ABSTRACT

The present invention relates to methods and biomarkers for detection and characterization of lymphoma (e.g., splenic marginal zone lymphoma) in biological samples (e.g., tissue samples, blood samples, plasma samples, cell samples, serum samples).

This application claims priority to U.S. Provisional Patent Application No. 61/666,445, filed Jun. 29, 2012, which is herein incorporated by reference in its entirety.

### STATEMENT REGARDING FEDERALLY SPONSORED RESEARCH OR DEVELOPMENT

This invention was made with government support under DE019249, CA136905 and CA140806 awarded by the National Institutes of Health. The government has certain rights in the invention.

### FIELD OF THE INVENTION

The present invention relates to methods and biomarkers for detection and characterization of lymphoma (e.g., splenic marginal zone lymphoma) in biological samples (e.g., tissue samples, blood samples, plasma samples, cell samples, serum samples).

### BACKGROUND OF THE INVENTION

Splenic marginal zone lymphoma (SMZL) is an indolent malignancy of splenic B lymphocytes characterized by splenomegaly, peripheral leukocytosis and cytopenias with a median age of onset of greater than 50 years. SMZL is the most common primary malignancy of the spleen and represents approximately 10% of all lymphomas that involve the spleen (Franco et al., 2003 Blood 101:2464-2472).

Although the disease course is usually indolent, with many patients surviving beyond 10 years, some patients present with more aggressive disease and survival between 1 and 2 years (Chacon et al., 2002 Blood 100:1648-1654). A “watch and wait” approach to instituting therapy may be considered for patients with favorable clinical prognostic factors (Arcaini et al., 2006) however, as it is difficult to predict subsequent risk of disease aggressiveness or refractoriness, a common first-line therapeutic approach is splenectomy and anti-Blymphocyte biological agents such as the anti-CD20 antibody (rituximab). Refractory cases may then be treated with more toxic chemotherapies including alkylating agents or purine analogs. In contrast to many other B-cell malignancies, SMZL is not associated with recurrent balanced translocations or genetic mutations. Moreover, little is known about the genetic events underpinning the development of aggressive or refractory disease or the transformation to higher-grade disease.

Better, more effective non-invasive tests for early detection of lymphomas are needed to lower the morbidity and mortality associated with such cancers.

### SUMMARY OF THE INVENTION

The present invention relates to methods and biomarkers for detection and characterization of lymphoma (e.g., splenic marginal zone lymphoma) in biological samples (e.g., tissue samples, blood samples, plasma samples, cell samples, serum samples).

For example, in some embodiments, the present invention provides a method for detecting NOTCH2 variants associated with splenic marginal zone lymphoma (SMZL) in a subject, comprising: a) contacting a sample from a subject with a NOTCH2 variant detection assay under conditions that the presence of a NOTCH variant associated with SMZL is determined; and b) diagnosing SMZL in the subject when the NOTCH2 variants are present in the sample. In some embodiments, the NOTCH2 variant encodes a loss of function mutation. In some embodiments, the loss of function mutation is a truncation mutation (e.g., the truncation results in a non-functional PEST domain of the NOTCH2 polypeptide). The present invention is not limited to a particular NOTCH2 mutation. Examples include, but are not limited to, one or more of c.6909dupC (p.I2304fsX9), c.7198C>T (p.R2400X), c.4999G>A (p.V1667I), c.6304A>T (p.K2102X), c.6824C>A (p.A2275D), c.6834delinsGCACG (p.T2280fsX12), c.6853C>T (p.Q2285X), c.6868G>A (p.E2290X), c.6873delG (p.K2292fsX3), c.6909delC (p.I2304fsX2), c.6909delC (p.I2304fsX2) plus c.7072A>G (p.M2358V), c.6909dupC (p.I2304fsX9), c.6910delinsCCC (p.I2304fsX3), c.6973C>T (p.Q2325X), or c.7231G>T (p.E2411X). In some embodiments, variants in additional genes are detected in combination with the described NOTCH2 variants (e.g., those described in Tables 5 and 6). In some embodiments, the detection assay is a variant NOTCH2 nucleic acid or polypeptide detection assay. In some embodiments, detecting variant NOTCH2 nucleic acids comprises one or more nucleic acid detection methods selected from, for example, sequencing, amplification or hybridization. In some embodiments, the biological sample is a tissue sample, a cell sample, or a blood sample. In some embodiments, the determining comprises a computer implemented method (e.g., analyzing NOTCH2 variant information and displaying the information to a user). In some embodiments, the method further comprises the step of treating the subject for SMZL and monitoring the subject for the presence of NOTCH2 variants associated with SMZL. In some embodiments, the method further comprises the step of treating the subject for SMZL under conditions such that one or more symptoms of SMZL are decreased or eliminated. Additional embodiments provide the use of a variant NOTCH2 nucleic acid or polypeptide for detecting SMZL in a subject.

In still further embodiments, the present invention provides a method of determining a decreased time to adverse outcome in a subject diagnosed with SMZL, comprising: a) contacting a sample from a subject with a NOTCH2 variant detection assay under conditions that the presence of a NOTCH2 variant associated with SMZL is determined; and b) diagnosing a decreased time to adverse outcome in the subject when the NOTCH2 variants are present in the sample. In some embodiments, the adverse outcome is relapse of SMZL, metastasis, or death.

Additional embodiments will be apparent to persons skilled in the relevant art based on the teachings contained herein.

### DESCRIPTION OF THE DRAWINGS

FIG. 1 shows whole genome sequencing identifing NOTCH2 mutations in SMZL. Panel A shows a representative case of SMZL with typical histopathological features of

SMZL including expansion of pale staining marginal zones surrounding splenic follicles in a biphasic pattern. Panels B and C display reverse complement sequence reads (Read Alignment) mapped to the reference genome (Reference Sequence) from two of three index samples with mutations in NOTCH2 (boxed) with deviations from reference genome highlighted in blue. Bottom panel shows Sanger sequencing electropherograms confirming mutations in the index cases (SMZL) and the absence of the mutations in matched normal constitutional tissue (Germline).

FIG. 2 shows the discovery, validation and specificity assessment of NOTCH2 mutations in SMZL and other B-cell lymphomas. A summary of the experimental design and results illustrates initial NOTCH2 mutation discovery in three of six index SMZL cases through whole genome sequencing, all of which were confirmed as somatic mutations by traditional Sanger sequencing.

FIG. 3 shows NOTCH2 mutations in SMZL. Upper Panel: The 34 exons of NOTCH2 are shown as grey boxes flanked by the 5′- and 3′-untranslated (UTR) regions of exons 1 and 34, respectively, above the protein domain structure of NOTCH2 including 36 epidermal growth factor-like repeats (EGFR; mediates ligand binding), three Lin-12-NOTCH repeat (LNR) domains (prevents ligand independent activation), the heterodimerization domain (HD; prevents ligand-independent activation), a single-pass transmembrane region (TM), RBP-J kappa-associated module domain (RAM; required for NOTCH signaling), six ankyrin repeats (AR; bind the CSL transcription factor), the transactivation domain (TAD), and the proline-, glutamate-, serine- and threonine-rich domain (PEST). Middle Panel: Three mutations in the TAD and the PEST domain downstream of the AR region were identified in the SMZL discovery cohort. Lower Panel: Targeted Sanger sequencing of the SMZL validation cohort uncovered the same as well as additional missense (triangles), non-sense and frameshift (circles) mutations in the HD, TAD and PEST domains.

FIG. 4 shows that NOTCH2 mutations lead to increased NOTCH activity. NOTCH2 mutants were prepared using a construct lacking the EGF domain region (ΔEGF) and expressed in 293T cells.

FIG. 5. Impact of NOTCH2 mutations on clinical outcome in SMZL. Panel A displays the frequency of NOTCH2 mutations in SMZL, MALT and other B-cell proliferative disorders divided among the different domains of the NOTCH2 protein. Panel B displays the cumulative probability of relapse, transformation or death from time of tissue diagnosis for patients with NOTCH2-mutated and NOTCH2-wild-type SMZL. Panel C displays the relapse-free survival from tissue diagnosis.

FIG. 6 shows an additional index case with c.7198C>T (p.R2400X) mutation identified by genome sequencing.

FIG. 7 shows sanger sequencing identification of NOTCH2 mutations in SMZL validation cohort.

FIG. 8 shows NOTCH1 and NOTCH2 mutations in COSMIC database.

FIG. 9 shows the impact of NOTCH2 mutations on overall survival in SMZL.

FIG. 10 shows NOTCH2 mutations in Hajdu-Cheney Syndrome.

FIG. 11 shows structural alterations in index SMZL cases.

### DEFINITIONS

To facilitate an understanding of the present invention, a number of terms and phrases are defined below:

As used herein, the term “sensitivity” is defined as a statistical measure of performance of an assay (e.g., method, test), calculated by dividing the number of true positives by the sum of the true positives and the false negatives.

As used herein, the term “specificity” is defined as a statistical measure of performance of an assay (e.g., method, test), calculated by dividing the number of true negatives by the sum of true negatives and false positives.

As used herein, the term “informative” or “informativeness” refers to a quality of a marker or panel of markers, and specifically to the likelihood of finding a marker (or panel of markers) in a positive sample.

As used herein, the term “metastasis” is meant to refer to the process in which cancer cells originating in one organ or part of the body relocate to another part of the body and continue to replicate. Metastasized cells subsequently form tumors which may further metastasize. Metastasis thus refers to the spread of cancer from the part of the body where it originally occurs to other parts of the body.

The term “neoplasm” as used herein refers to any new and abnormal growth of tissue. Thus, a neoplasm can be a premalignant neoplasm or a malignant neoplasm. The term “neoplasm-specific marker” refers to any biological material that can be used to indicate the presence of a neoplasm. Examples of biological materials include, without limitation, nucleic acids, polypeptides, carbohydrates, fatty acids, cellular components (e.g., cell membranes and mitochondria), and whole cells. The term “SMZL-specific marker” refers to any biological material that can be used to indicate the presence of SMZL. Examples of SMZL specific markers include, but are not limited to, the NOTCH2 variants described herein.

As used herein, the term “adverse outcome” refers to an undesirable outcome in a patient diagnosed with SMZL. In some embodiments, the patient is undergoing or has undergone treatment for SMZL. Examples of adverse outcome include but are not limited to, recurrence of SMZL, metastasis, transformation, or death.

As used herein, the term “amplicon” refers to a nucleic acid generated using primer pairs. The amplicon is typically single-stranded DNA (e.g., the result of asymmetric amplification), however, it may be RNA or dsDNA.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded.

If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

The term “locus” as used herein refers to a nucleic acid sequence on a chromosome or on a linkage map and includes the coding sequence as well as 5′ and 3′ sequences involved in regulation of the gene.

### DETAILED DESCRIPTION OF THE INVENTION

The present invention relates to methods and biomarkers for detection and characterization of lymphoma (e.g., splenic marginal zone lymphoma) in biological samples (e.g., tissue samples, blood samples, plasma samples, cell samples, serum samples).

The NOTCH family of transmembrane receptor proteins is important for mediating cell fate determination and differentiation in a variety of embryonic and adult tissues. During hematopoietic differentiation, NOTCH1 signaling is known to influence cell-fate decisions as lymphocytes differentiate into B- or T-cells (Pui et al., 1999 Immunity 11:299-308; Radtke et al., 1999 Immunity 10:547-558; Robey and Bluestone, 2004 Curr Opin Immunol 16:360-366). Moreover, NOTCH2 is known to control B-lymphocyte specification into cells of marginal zone lineage (Pillai and Cariappa, 2009 Nat Rev Immunol 9:767-777). Whereas defects in NOTCH1 signaling have been implicated in oncogenesis in acute T-lymphoblastic leukemia (Aster et al., 2011 J Pathol 223:262-273; Weng et al., 2004 Science 306:269-271), chronic lymphocytic leukemia/small lymphocytic lymphoma (Del Giudice et al., 2012 Haematologica 97:437-441.; Puente et al., 2011) and mantle cell lymphoma (Kridel et al., 2012 Blood 119:1963-1971), comparatively little is known about the potential role of NOTCH2 signaling defects in the development of malignancies affecting cells of B-lymphocyte lineage (Aster et al., 2011 J Pathol 223:262-273).

Experiments conducted during the course of development of embodiments of the present invention utilized whole genome and targeted Sanger gene sequencing to identify recurrent mutations predominantly clustered in the C-terminal portion of the NOTCH2 gene in SMZL. NOTCH2 mutations were identified in half of these cases. Sanger sequencing of 93 additional SMZLs and 103 other types of B-cell lymphoma or leukemia or reactive lymphoid hyperplasia showed NOTCH2 mutations in 22 additional SMZL patients, yielding an overall frequency of 25.3%. No mutations were identified in other non-MZL B-cell lymphomas and leukemias analyzed. Moreover, in 19 patients with NOTCH2-mutated SMZL constitutional DNA was available for assessment and was confirmed to be wild-type indicating somatic acquisition of NOTCH2 mutation in SMZL.

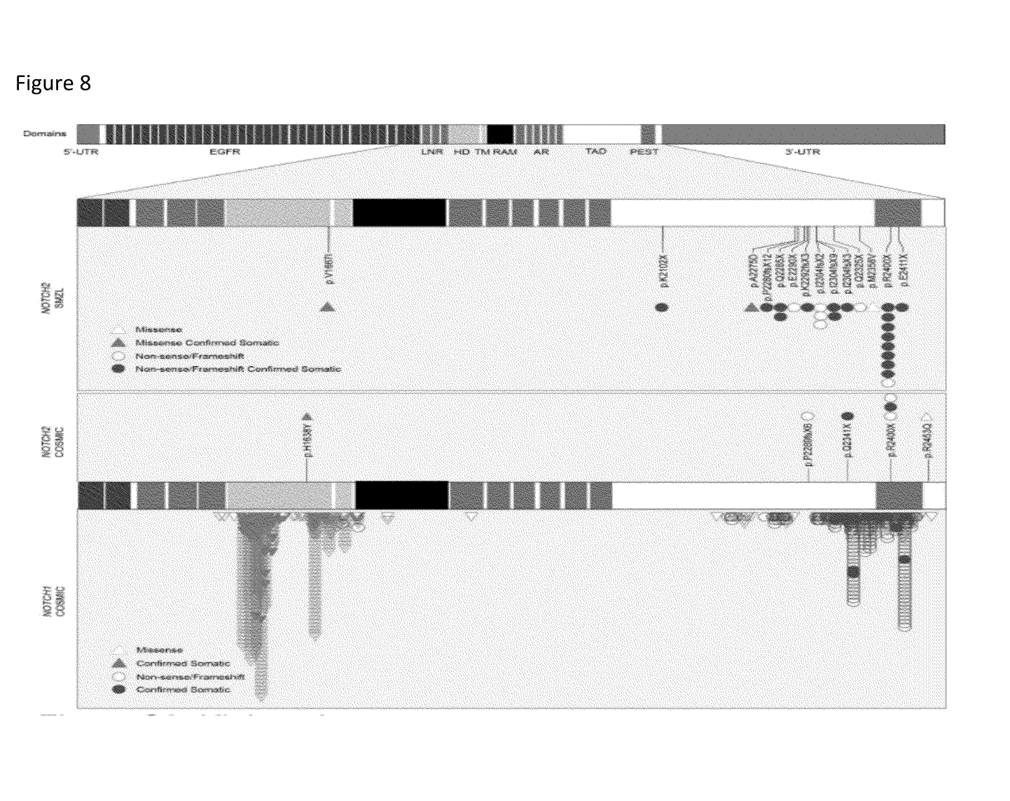

In total, 26 NOTCH2 mutations were identified in 25 SMZL patients. These mutations represented six unique types of non-sense mutations, five unique types of frameshift mutations and three unique types of missense mutations. Twenty-five of these mutations affected the TAD or PEST domains with 23 predicted to yield protein truncation at or upstream of the PEST domain. The remaining case harbored a somatic p.V1667I mutation in the HD. All of these mutations were identified in the same protein domains as have been reported for NOTCH1 in T-ALL, CLL/SLL and MCL. However, NOTCH1 mutations in T-ALL are more prevalent in the HD than the TAD and PEST domain (FIG. 8). Disruption of the C-terminal PEST domain renders NOTCH less susceptible to regulation by ubiquitin-mediated proteolysis and thus results in increased activation of the NOTCH pathway (Gupta-Rossi et al., 2001 JBiol Chem 276:34371-34378; Oberg et al., 2001 J Biol Chem 276:35847-35853; Wu et al., 2001 Mol Cell Biol 21:7403-7415). Using reporter assays for assessment of NOTCH activation, it was confirmed that representative mutations affecting either the PEST or HD indeed resulted in NOTCH2 transcriptional hyperactivation.

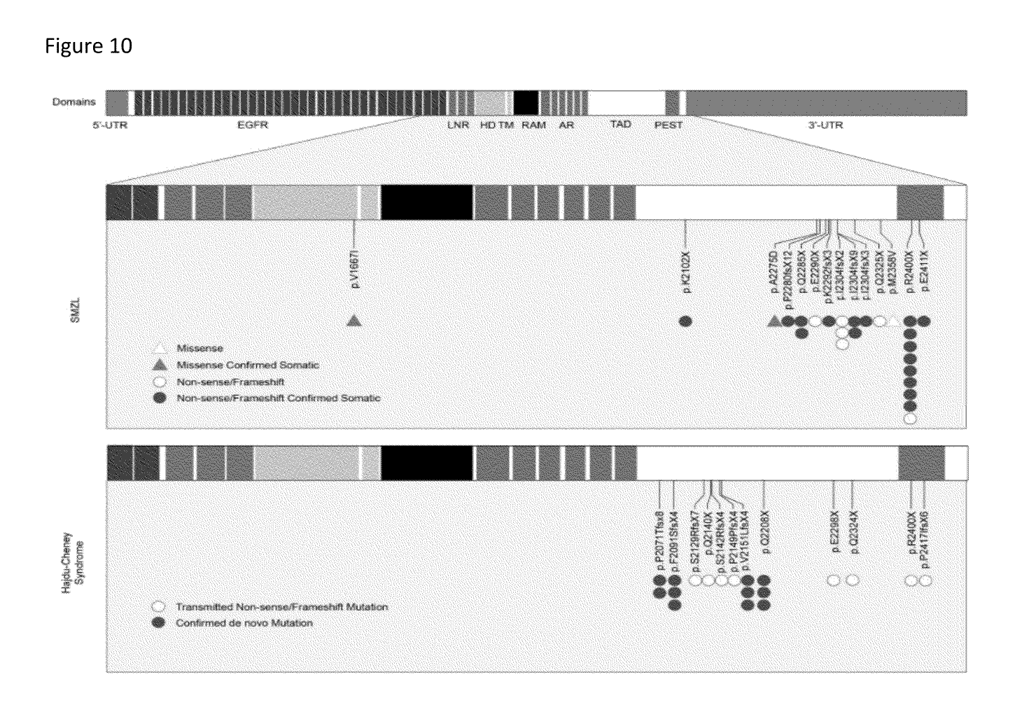

Pathogenic germline mutations in the TAD/PEST domain of NOTCH2 have been reported in Hajdu-Cheney syndrome (HCS), a rare autosomal dominant skeletal disorder characterized by facial anomalies, acro-osteolysis and osteoporosis (Isidor et al., 2011 Nat Genet 43:306-308; Simpson et al., 2011 Nat Genet 43:303-305). The NOTCH2 mutations in HCS include one report of a transmitted p.R2400X mutation (Simpson et al., 2011 supra) (FIG. 10). With regard to neoplasia, isolated NOTCH2 mutations have been reported in a single case of SMZL and a single case of MZL in a previous study (Troen et al., 2008 Haematologica 93:1107-1109) as well as a small number of cases of diffuse large B-cell lymphoma (Lee et al., 2009 Cancer Sci 100:920-926), but no evidence for prognostic implications was presented in either study. NOTCH2 shares significant homology with NOTCH1 and transforming capacity has been demonstrated for truncated alleles of both proteins (Capobianco et al., 1997 Mol Cell Biol 17:6265-6273; Ellisen et al., 1991 Cell 66:649-661; Rohn et al., 1996 J Virol 70:8071-8080). Loss-of-function mutations affecting NOTCH family and pathway genes have recently been implicated in the pathogenesis of myeloid (Klinakis et al., 2011 Nature 473:230-233) and epithelial malignancies (Agrawal et al., 2011 Science 333:1154-1157; Mazur et al., 2010 Proc Natl Acad Sci USA 107:13438-13443; Stransky et al., 2011 Science 333:1157-1160; Viatour et al., 2011 J Exp Med 208:1963-1976; Wang et al., 2011 Proc Natl Acad Sci USA 108:17761-17766) and neuroblastoma (Zage et al., 2012 Pediatr Blood Cancer 58:682-689). These studies highlight the context-dependent roles of NOTCH and its signaling partners, which upon mutation, may contribute to the pathogenesis of neoplasia via different mechanisms in diverse cell types. Altogether, these findings indicate that the 26 NOTCH2 mutations identified are pathogenic events contributing to aberrant NOTCH2 signaling in malignant SMZL cells.

Examination of NOTCH2 mutational status in non-splenic MZLs revealed mutation in approximately 5% of cases analyzed. The NOTCH2 mutation identified in a single case of extranodal MZL of the breast was a p.R2400X nonsense mutation. This mutation was also identified in nine of 99 (9.1%) SMZL cases. The selectivity of NOTCH2 mutations for malignancies of marginal zone B-cells is in keeping with the known role of NOTCH2 in marginal zone cell fate determination (Saito et al., 2003 Immunity 18:675-685; Witt et al., 2003 J Immunol 171:2783-2788). It is noteworthy that NOTCH1 dictates T-cell fate and supra-physiological NOTCH1 signaling induces T-ALL (Weng et al., 2004 Science 306:269-271). The present invention is not limited to a particular mechanism. Indeed, an understanding of the mechanism is not necessary to practice the present invention. Nonetheless, it is contemplated that since NOTCH2 specifies marginal zone-B cell fate, supra-physiological NOTCH2 signaling plays a role in pathogenesis of MZL. Somatic mutations affecting specific genes that impact SMZL prognosis are largely unknown. While previous studies have implicated a role for mutations targeting genes in the NFKB pathway in a subset of SMZL (Rossi et al., 2011 Blood 118:4930-4934), only TP53 alterations present in a small minority of cases has been demonstrated to impact SMZL prognosis (Rinaldi et al., 2011 Blood 117:1595-1604; Salido et al., 2010 Blood 116:1479-1488). Experiments described herein found that the presence of NOTCH2 mutation in SMZLs at time of diagnosis predicted an adverse disease course characterized either by refractoriness to therapy, histological transformation to higher grade disease, or an otherwise aggressive clinical course. Assessment of NOTCH2 mutation status in cases of SMZL is thus useful to predict risk of aggressive disease and inform clinical decision-making at diagnosis, with the presence of NOTCH2 mutation being an indication for more aggressive therapy.

### Diagnostic and Screening Applications

Embodiments of the present invention provide diagnostic, prognostic, and screening methods. In some embodiments, methods characterize and diagnose lymphoma (e.g., splenic marginal zone lymphoma (SMZL) or non-splenic MZLs). Exemplary, non-limiting methods of identifying NOTCH2 mutations are described below.

### A. NOTCH2 Mutations

Embodiments of the present invention provide compositions and methods for detecting mutations in NOTCH2 (e.g., to identify or diagnose splenic lymphomas). The present invention is not limited to particular NOTCH2 mutations. In some embodiments, mutations are loss of function mutations (e.g., truncation, nonsense, missense, or frameshift mutations).

Exemplary mutations include, but are not limited to, c.6909dupC (p.I2304fsX9), c.7198C>T (p.R2400X), c.4999G>A (p.V1667I), c.6304A>T (p.K2102X), c.6824C>A (p.A2275D), c.6834delinsGCACG (p.T2280fsX12), c.6853C>T (p.Q2285X), c.6868G>A (p.E2290X), c.6873delG (p.K2292fsX3), c.6909delC (p.I2304fsX2), c.6909delC (p.I2304fsX2) plus c.7072A>G (p.M2358V), c.6909dupC (p.I2304fsX9), c.6910delinsCCC (p.I2304fsX3), c.6973C>T (p.Q2325X), or c.7231G>T (p.E2411X).

While the present invention exemplifies several markers specific for detecting splenic lymphoma, any marker that is correlated with the presence or absence or prognosis of splenic lymphomas may be used. A marker, as used herein, includes, for example, nucleic acid(s) whose production or mutation or lack of production is characteristic of a splenic lymphoma and mutations that cause the same effect (e.g., deletions, truncations, etc).

In some embodiments, one or more (e.g., 2, 3, 4, 5, 6, 7, 8, 9, 10, 15, 20, 25, or more (e.g., all)) of the mutations are identified in order to diagnose or characterize splenic lymphoma. In some embodiments, mutations are identified in combination with one or more additional markers of splenic lymphomas or other cancers (e.g., those described in Tables 5 and 6). In some embodiments, multiple markers are detected in a panel or multiplex format.

Particular combinations of markers may be used that show optimal function with different ethnic groups or sex, different geographic distributions, different stages of disease, different degrees of specificity or different degrees of sensitivity. Particular combinations may also be developed which are particularly sensitive to the effect of therapeutic regimens on disease progression. Subjects may be monitored after a therapy and/or course of action to determine the effectiveness of that specific therapy and/or course of action.

### B. Detection of NOTCH2 Alleles

In some embodiments, the present invention provides methods of detecting the presence of wild type or variant (e.g., mutant or polymorphic) NOTCH2 nucleic acids or polypeptides. The detection of mutant NOTCH2 finds use in the diagnosis of disease (e.g., splenic lymphomas), research, and selection of appropriate treatment and/or monitoring regimens.

Accordingly, the present invention provides methods for determining whether a patient has a NOTCH2 mutation profile associated with a splenic lymphoma.

A number of methods are available for analysis of variant (e.g., mutant or polymorphic) nucleic acid sequences. Assays for detecting variants (e.g., polymorphisms or mutations) fall into several categories, including, but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays are useful in the present invention.

Any patient sample containing NOTCH2 nucleic acids or polypeptides may be tested according to the methods of the present invention. By way of non-limiting examples, the sample may be tissue, blood, urine, semen, or a fraction thereof (e.g., plasma, serum, whole blood, spleen cells, etc.).

The patient sample may undergo preliminary processing designed to isolate or enrich the sample for the NOTCH2 nucleic acids or polypeptides or cells that contain NOTCH2. A variety of techniques known to those of ordinary skill in the art may be used for this purpose, including but not limited: centrifugation; immunocapture; cell lysis; and, nucleic acid target capture (See, e.g., EP Pat. No. 1 409 727, herein incorporated by reference in its entirety).

i. DNA and RNA Detection

The NOTCH2 variants of the present invention may be detected as genomic DNA or mRNA using a variety of nucleic acid techniques known to those of ordinary skill in the art, including but not limited to: nucleic acid sequencing; nucleic acid hybridization; and, nucleic acid amplification.

1. Sequencing

Illustrative non-limiting examples of nucleic acid sequencing techniques include, but are not limited to, chain terminator (Sanger) sequencing and dye terminator sequencing.

Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, fluorescent or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide.

Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

Some embodiments of the present invention utilize next generation or high-throughput sequencing. A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

In some embodiments, sequencing technology including, but not limited to, pyrosequencing, sequencing-by-ligation, single molecule sequencing, sequence-by-synthesis

(SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the technology finds use in automated sequencing techniques understood in that art. In some embodiments, the present technology finds use in parallel sequencing of partitioned amplicons (PCT Publication No: WO2006084132 to Kevin McKernan et al., herein incorporated by reference in its entirety). In some embodiments, the technology finds use in DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbial., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbial., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbial., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, the technology finds use in nanopore sequencing (see, e.g., Astier et al., J. Am. Chem. Soc. 2006 Feb 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined

In certain embodiments, the technology finds use in HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜400 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

The technology finds use in another nucleic acid sequencing approach developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. Pat. App. Ser. No. 11/671956; U.S. Pat. App. Ser. No. 11/781166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

In some embodiments, capillary electrophoresis (CE) is utilized to analyze amplification fragments. During capillary electrophoresis, nucleic acids (e.g., the products of a PCR reaction) are injected electrokinetically into capillaries filled with polymer. High voltage is applied so that the fluorescent DNA fragments are separated by size and are detected by a laser/camera system. In some embodiments, CE systems from Life Technogies (Grand Island, N.Y.) are utilized for fragment sizing (See e.g., U.S. Pat. No. 6,706,162, U.S. Pat. No. 8,043,493, each of which is herein incorporated by reference in its entirety).

2. Hybridization

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

3. Microarrays

In some embodiments, microarrays are utilized for detection of NOTCH2 nucleic acid sequences. Examples of microarrays include, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Arrays can also be used to detect copy number variations at al specific locus. These genomic micorarrys detect microscopic deletions or other variants that lead to disease causing alleles.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

4. Amplification

NOTCH2 nucleic acid may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary

DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPaS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, DC (1993)).

5. Detection Methods

Non-amplified or amplified NOTCH2 nucleic acids can be detected by any conventional means. For example, nucleic acid can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence.

By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

ii. Detection of Variant NOTCH2 Proteins

In other embodiments, variant NOTCH2 polypeptides are. Any suitable method may be used to detect truncated or mutant NOTCH2 polypeptides including, but not limited to, those described below.

1. Antibody Binding

In some embodiments, antibodies (See below for antibody production) are used to determine if an individual contains an allele encoding a variant NOTCH2 polypeptide. In preferred embodiments, antibodies are utilized that discriminate between variant (i.e., truncated proteins); and wild-type proteins. In some embodiments, the antibodies are directed to the C-terminus of NOTCH2 proteins. Proteins that are recognized by the N-terminal, but not the C-terminal antibody are truncated. In some embodiments, quantitative immunoassays are used to determine the ratios of C-terminal to N-terminal antibody binding. In other embodiments, identification of variants of NOTCH2 is accomplished through the use of antibodies that differentially bind to wild type or variant forms of NOTCH2 proteins.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Patents 5,885,530, 4,981,785, 6,159,750, and 5,358,691, each of which is herein incorporated by reference. In some embodiments, the analysis and presentation of results is also automated. For example, in some embodiments, software that generates a prognosis based on the result of the immunoassay is utilized. In other embodiments, the immunoassay described in U.S. Pat. Nos. 5,599,677 and 5,672,480; each of which is herein incorporated by reference.

C. Kits for Detecting NOTCH2 Mutant or Variant Alleles

The present invention also provides kits for determining whether an individual contains a wild-type or variant (e.g., mutant or polymorphic) allele of NOTCH2. In some embodiments, the kits are useful for determining whether the subject has a splenic lymphoma (e.g., SMZL) or to provide a prognosis to an individual diagnosed with a splenic lymphoma (e.g., SMZL). The diagnostic kits are produced in a variety of ways. In some embodiments, the kits contain at least one reagent useful, necessary, or sufficient for specifically detecting a mutant or variant NOTCH2 allele or protein. In some embodiments, the kits contain reagents for detecting a truncation in the NOTCH2 polypeptide. In preferred embodiments, the reagent is a nucleic acid that hybridizes to nucleic acids containing the mutation and that does not bind to nucleic acids that do not contain the mutation. In other embodiments, the reagents are primers for amplifying the region of DNA containing the mutation. In still other embodiments, the reagents are antibodies that preferentially bind either the wild-type or truncated or variant NOTCH2 proteins.

In some embodiments, the kits include ancillary reagents such as buffering agents, nucleic acid stabilizing reagents, protein stabilizing reagents, and signal producing systems (e.g., florescence generating systems as Fret systems), and software (e.g., data analysis software). The test kit may be packages in any suitable manner, typically with the elements in a single container or various containers as necessary along with a sheet of instructions for carrying out the test. In some embodiments, the kits also preferably include a positive control sample.

In some embodiments, markers (e.g., those described herein) are detected alone or in combination with other markers in a panel or multiplex format. For example, in some embodiments, a plurality of markers are simultaneously detected in an array or multiplex format (e.g., using the detection methods described herein).

D. Bioinformatics

For example, in some embodiments, a computer-based analysis program is used to translate the raw data generated by the detection assay (e.g., the presence, absence, or amount of a given NOTCH2 allele or polypeptide) into data of predictive value for a clinician. The clinician can access the predictive data using any suitable means. Thus, in some preferred embodiments, the present invention provides the further benefit that the clinician, who may not be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information providers, medical personal, and subjects. For example, in some embodiments of the present invention, a sample (e.g., a biopsy or a blood or serum sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., presence of wild type or mutant NOTCH2), specific for the screening, diagnostic or prognostic information desired for the subject.

The profile data is then prepared in a format suitable for interpretation by a treating clinician. For example, rather than providing raw data, the prepared format may represent a diagnosis or risk assessment (e.g., diagnosis or prognosis of SMZL) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

In some embodiments, the methods disclosed herein are useful in monitoring the treatment of lymphoma (e.g., SMZL). For example, in some embodiments, the methods may be performed immediately before, during and/or after a treatment to monitor treatment success. In some embodiments, the methods are performed at intervals on disease free patients to ensure treatment success.

The present invention also provides a variety of computer-related embodiments. Specifically, in some embodiments the invention provides computer programming for analyzing and comparing a pattern of SMZL-specific marker detection results in a sample obtained from a subject to, for example, a library of such marker patterns known to be indicative of the presence or absence of SMZL, or a particular stage or prognosis of SMZL.

In some embodiments, the present invention provides computer programming for analyzing and comparing a first and a second pattern of SMZL-specific marker detection results from a sample taken at least two different time points. In some embodiments, the first pattern may be indicative of a pre-cancerous condition and/or low risk condition for SMZL cancer and/or progression from a pre-cancerous condition to a cancerous condition. In such embodiments, the comparing provides for monitoring of the progression of the condition from the first time point to the second time point. In yet another embodiment, the invention provides computer programming for analyzing and comparing a pattern of SMZL-specific marker detection results from a sample to a library of SMZL-specific marker patterns known to be indicative of the presence or absence of a SMZL, wherein the comparing provides, for example, a differential diagnosis between an aggressively malignant SMZL cancer and a less aggressive SMZL cancer (e.g., the marker pattern provides for staging and/or grading of the cancerous condition).

The methods and systems described herein can be implemented in numerous ways. In one embodiment, the methods involve use of a communications infrastructure, for example the internet. Several embodiments of the invention are discussed below. It is also to be understood that the present invention may be implemented in various forms of hardware, software, firmware, processors, distributed servers (e.g., as used in cloud computing) or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

For example, during or after data input by the user, portions of the data processing can be performed in the user-side computing environment. For example, the user-side computing environment can be programmed to provide for defined test codes to denote platform, carrier/diagnostic test, or both; processing of data using defined flags, and/or generation of flag configurations, where the responses are transmitted as processed or partially processed responses to the reviewer's computing environment in the form of test code and flag configurations for subsequent execution of one or more algorithms to provide a results and/or generate a report in the reviewer's computing environment.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which generally includes test data (e.g., specific gene products assayed), and test result data (e.g., the pattern of gastrointestinal neoplasm-specific marker detection results from a sample). This information received can be stored at least temporarily in a database, and data analyzed in comparison to a library of marker patterns known to be indicative of the presence or absence of a pre-cancerous condition, or known to be indicative of a stage and/or grade of gastrointestinal cancer.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In some embodiments, all or a portion of the input data and/or all or a portion of the output data (e.g., usually at least the library of the pattern of gastrointestinal neoplasm-specific marker detection results known to be indicative of the presence or absence of a pre-cancerous condition) are maintained on a server for access, e.g., confidential access. The results may be accessed or sent to professionals as desired.

A system for use in the methods described herein generally includes at least one computer processor (e.g., where the method is carried out in its entirety at a single site) or at least two networked computer processors (e.g., where detected marker data for a sample obtained from a subject is to be input by a user (e.g., a technician or someone performing the assays)) and transmitted to a remote site to a second computer processor for analysis (e.g., where the pattern of SMZL-specific marker) detection results is compared to a library of patterns known to be indicative of the presence or absence of a pre-cancerous condition), where the first and second computer processors are connected by a network, e.g., via an intranet or internet). The system can also include a user component(s) for input; and a reviewer component(s) for review of data, and generation of reports, including detection of a pre-cancerous condition, staging and/or grading of SMZL, or monitoring the progression of a pre-cancerous condition or SMZL. Additional components of the system can include a server component(s); and a database(s) for storing data (e.g., as in a database of report elements, e.g., a library of marker patterns known to be indicative of the presence or absence of a pre-cancerous condition and/or known to be indicative of a grade and/or a stage of SMZL, or a relational database (RDB) which can include data input by the user and data output. The computer processors can be processors that are typically found in personal desktop computers (e.g., IBM, Dell, Macintosh), portable computers, mainframes, minicomputers, tablet computer, smart phone, or other computing devices.

The input components can be complete, stand-alone personal computers offering a full range of power and features to run applications. The user component usually operates under any desired operating system and includes a communication element (e.g., a modem or other hardware for connecting to a network using a cellular phone network, Wi-Fi, Bluetooth,

Ethernet, etc.), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe, or distributed across multiple servers (e.g., as in cloud computing applications) and offers data management, information sharing between clients, network administration and security. The application and any databases used can be on the same or different servers. Other computing arrangements for the user and server(s), including processing on a single machine such as a mainframe, a collection of machines, or other suitable configuration are contemplated. In general, the user and server machines work together to accomplish the processing of the present invention.

Where used, the database(s) is usually connected to the database server component and can be any device which will hold data. For example, the database can be any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

In certain embodiments, the present invention provides methods for obtaining a subject's risk profile for developing SMZL or having aggressive SMZL. In some embodiments, such methods involve obtaining a blood or blood product sample from a subject (e.g., a human at risk for developing SMZL; a human undergoing a routine physical examination, or a human diagnosed with SMZL), detecting the presence or absence of the NOTCH2 variants described herein associated SMZL in the sample, and generating a risk profile for developing SMZL or progressing to metastatic or aggressive SMZL. For example, in some embodiments, a generated profile will change depending upon specific markers and detected as present or absent or at defined threshold levels. The present invention is not limited to a particular manner of generating the risk profile. In some embodiments, a processor (e.g., computer) is used to generate such a risk profile. In some embodiments, the processor uses an algorithm (e.g., software) specific for interpreting the presence and absence of specific exfoliated epithelial markers as determined with the methods of the present invention. In some embodiments, the presence and absence of specific NOTCH2 variants as determined with the methods of the present invention are imputed into such an algorithm, and the risk profile is reported based upon a comparison of such input with established norms (e.g., established norm for pre-cancerous condition, established norm for various risk levels for developing SMZL, established norm for subjects diagnosed with various stages of SMZL cancer). In some embodiments, the risk profile indicates a subject's risk for developing SMZL or a subject's risk for re-developing SMZL. In some embodiments, the risk profile indicates a subject to be, for example, a very low, a low, a moderate, a high, and a very high chance of developing or re-developing SMZL cancer or having a poor prognosis (e.g., likelihood of long term survival) from SMZL. In some embodiments, a health care provider (e.g., an oncologist) will use such a risk profile in determining a course of treatment or intervention (e.g., biopsy, wait and see, referral to an oncologist, referral to a surgeon, etc.).

### EXPERIMENTAL

The following examples are provided in order to demonstrate and further illustrate certain preferred embodiments and aspects of the present invention and are not to be construed as limiting the scope thereof

### Example 1

### Material and Methods

Patients and samples. Six SMZL samples from the University of Michigan were selected as index cases for whole genome sequencing. To assess the prevalence of NOTCH2 mutations in SMZL, an additional 93 SMZL cases were obtained from The University of Texas MD Anderson Cancer Center (31 cases), the University of Utah Health Sciences Center (25 cases), the Southern California Permanente Medical Group (20 cases), the

University of Michigan (15 cases), and the University of Wisconsin (2 cases). Approval from the University of Michigan Hospital institutional review board (HUM0002325b) was obtained for these studies. In order to assess the specificity of NOTCH2 mutations in SMZL, genomic DNA was extracted from additional tissues representing non-SMZL diseases including 15 cases of chronic lymphocytic leukemia/small lymphocytic lymphoma (CLL/SLL), 15 cases of mantle cell lymphoma (MCL), 44 cases of grade 1-2 follicular lymphoma (FL), 15 cases of hairy cell leukemia (HCL) and 14 cases of reactive lymphoid hyperplasia (RLH). In addition, 19 cases of non-SMZL (e.g., nodal and extranodal/mucosa-associated lymphoid tissue lymphoma) were analyzed.

Pathological review. All specimens were reviewed independently and confirmed by consensus among three hematopathologists (MSL, NGB and KEJ) according to World Health Organization classification criteria without knowledge of NOTCH2 mutational status. Only cases containing adequate neoplastic tissue were included in subsequent analyses.

Whole genome and targeted NOTCH2 DNA sequencing. From each of six index SMZL cases, 10 μg of high-molecular-weight genomic DNA was extracted from fresh frozen tumor tissue using the QIAamp DNA extraction kit (QIAGEN) and subjected to whole genome sequencing by Complete Genomics, Incorporated (CGI; Mountain View, Calif.). CGI performs massively parallel short-read sequencing using a combinatorial probe-anchor ligation (cPAL) chemistry coupled with a patterned nanoarray-based platform of self-assembling DNA nanoballs (Drmanac et al., 2010 Science 327:78-81). Library generation, read-mapping to the NCBI reference genome (Build 37, RefSeq Accession numbers CM000663-CM00686), local de novo assembly and variant-calling protocols were performed as previously described (Drmanac et al., 2010 supra; Roach et al., 2010 Science 328:636-639). Initial read mapping and variant calling were performed using CGAtools v1.3.0. Additional downstream bioinformatic analyses were performed using custom designed PERL processing routines. Targeted sequencing of the NOTCH2 C-terminal coding exons 25 to 34 was performed using Sanger sequencing for the SMZL samples in the validation cohort. For all other samples, sequencing was confined to exons 26, 27 and 34 where all confirmed mutations in SMZL samples occurred. Somatic acquisition of each mutation was also assessed when matched constitutional tissue was available for analysis. Genomic DNA from index cases and genomic DNA corresponding to matched constitutional tissue were subjected to Sanger sequencing of regions of the NOTCH2 where mutations were observed through whole genome sequencing. For targeted sequencing of exons 25-34 in the NOTCH2 C-terminal region in the validation and specificity cohort samples, genomic DNA was extracted using both the QIAGEN BioRobot EZ1 and QIAamp FFPE DNA extraction kits (QIAGEN). For all Sanger sequencing reactions, PCR amplification was performed using Phusion DNA polymerase (New England Biolabs) followed by conventional Sanger sequencing technology using BigDye version 3.1 chemistry run on an Applied Biosystems 3730x1 DNA Sequencer at the University of Michigan DNA sequencing Core. All sequencing reactions were performed using nested sequencing primers. Sequencing trace analysis was performed using Mutation Surveyor software. All mutations were verified in at least two independent PCR amplification and sequencing reactions. cDNA nucleotide numbering of coding sequence is based on Genbank accession NG_{—}008163.1. Protein amino acid numbering is based on Genbank accession NP_{—}077719.2. Detailed primer sequences for targeted exon sequencing can be found in Table 1.

Cloning of NOTCH2 mutants and transactivation analysis. DNA constructs representing NOTCH2 mRNA lacking the EGF-repeat region (residues p.M1 to p.E1412, exons 1-24; ΔEGF) were engineered from full length NOTCH2 gene (OriGene; Rockville MD) to contain nucleotide sequence identical to that of wild-type and selected NOTCH2 mutations identified in index and validation SMZL samples. These constructs were transiently expressed in 293T cells and assessed for their ability to activate a NOTCH sensitive luciferase reporter gene system (SA Biosciences; Valencia Calif.). NOTCH2 mutations p.V1667I, p.Q2285X, p.I2304fsX9, p.R2400X and p.E2411X were introduced into ΔEGF NOTCH2 using QuickChange kit (Stratagene; La Jolla, CA) and appropriate truncation primers. Wild-type and NOTCH2 mutated constructs were cloned between EcoR1 and Xho1 restriction sites in the pCAGGS3.2 FLAG vector, which introduces FLAG tag at the N-terminal part of the protein. The sequence verified constructs were tested for expression of either wild-type or mutant NOTCH2 protein. NOTCH2 expression plasmids were introduced into 293T cells by transient transfection using Polyjet transfection reagent (SignaGen Laboratories; Rockville Md.) and assessed for their ability to activate a NOTCH-sensitive luciferase reporter gene, using Cignal RBP-Jk Reporter kit per protocol (SA Biosciences; Valencia Calif.). Briefly, cells in 24-well dishes were co-transfected in triplicate with 400 ng of various ΔEGF NOTCH2 expression constructs, a NOTCH-sensitive firefly luciferase reporter gene, and an internal control Renilla luciferase plasmid (Promega; Madison Wis.). Firefly luciferase activities were measured in whole-cell extracts prepared 48 h after transfection using the Dual Luciferase kit (Promega) and a specially configured luminometer (Berthold Technologies; Germany). Western blotting was performed using the extracts to ensure equal expression of different constructs. Briefly, 50 μl of total protein extracts from the reporter assay were separated on a high resolution SDS PAGE using SDS PAGE running buffer, followed by Western blotting using FLAG M2 mouse monoclonal antibody (Sigma-Aldrich; St. Louis Mo.).

Statistical analysis of clinical outcomes. Clinical outcomes data (time to transformation, relapse or death) were analyzed using standard survival analysis. Survival plots were generated using Kaplan-Meier method and Log-rank tests were used to compare survival times between patients with NOTCH2 mutations and patients with wild-type NOTCH2. Cox-proportional hazards regression analysis was conducted to compare the two groups of patients after adjusting for age, gender, performance status and stage at diagnosis. Statistical analyses were performed with SAS version 9.3.

### RESULTS

Genome Sequencing and NOTCH2 Mutation Confirmation

To gain insight into the pathogenesis of SMZL, WGS was performed on six index cases of SMZL. Whole genome sequencing (WGS) yielded an average of 350±10 million mapped reads per sample with an average of 97.6±0.08% genome coverage and 96.4±0.3% fully-called exome coverage. The median genomic sequencing depth exceeded 80× in all samples normalized across the entire genome. In order to enhance the ability to identify somatic alterations that are important in SMZL pathogenesis, variations that were present in any of the 6 SMZL genomes and not in the Database of SNPs (dbSNP) were investigated.

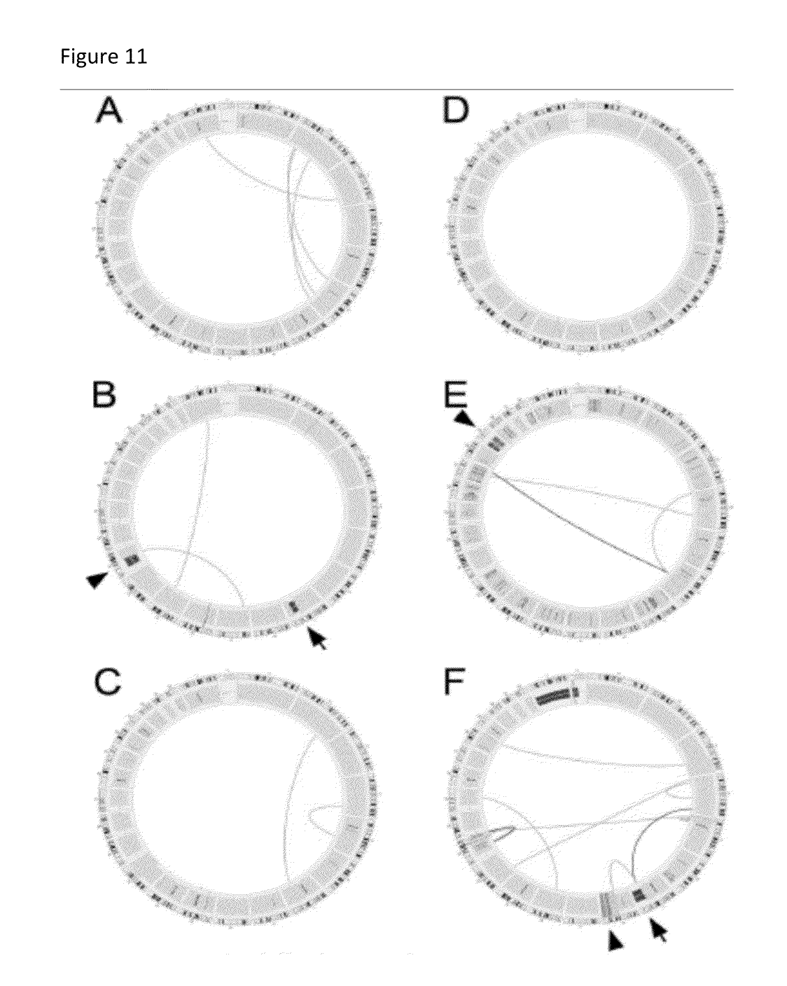

After normalization to publicly available constitutional normal genome sequencing data (Complete Genomics, Inc.), relative depth of coverage for distinct chromosomal regions were examined for evidence of recurrent chromosomal gains or losses. Corresponding plots of ploidy for each genome are shown in FIG. 11. Overall, the SMZL genomes had relatively few large structural alterations affecting chromosomes (FIG. 11). However, in keeping with previous observations (Gruszka-Westwood et al., 2003 Genes Chromosomes Cancer. 36:57-69; Mateo et al., 1999 Am J Pathol. 154:1583-1589; Rinaldi et al., 2011 Blood. 117:1595-1604; Salido et al., 2010 Blood. 116:1479-1488; Watkins et al., 2010 J Pathol. 220:461-474) recurrent deletions involving the long arm of chromosome 7 (del7q) were seen in two of the six index genomes (FIG. 11B and 11F, arrows). Additionally, one of these genomes also showed a partial loss of genetic elements corresponding to the sub-centromeric region of chromosome 13 (dell3q; FIG. 11B, arrowhead). Individual sequencing reads that mapped to two spatially separated regions of the reference genome were used to identify putative gene fusion or gene disruption events. To reduce the number of candidate structural alterations likely to be pathogenetic, these data were filtered to exclude structural alterations that did not affect coding elements of the involved gene(s) (FIG. 11). This analysis revealed no evidence of recurrent chromosomal translocation or chimeric fusions in the six index cases.

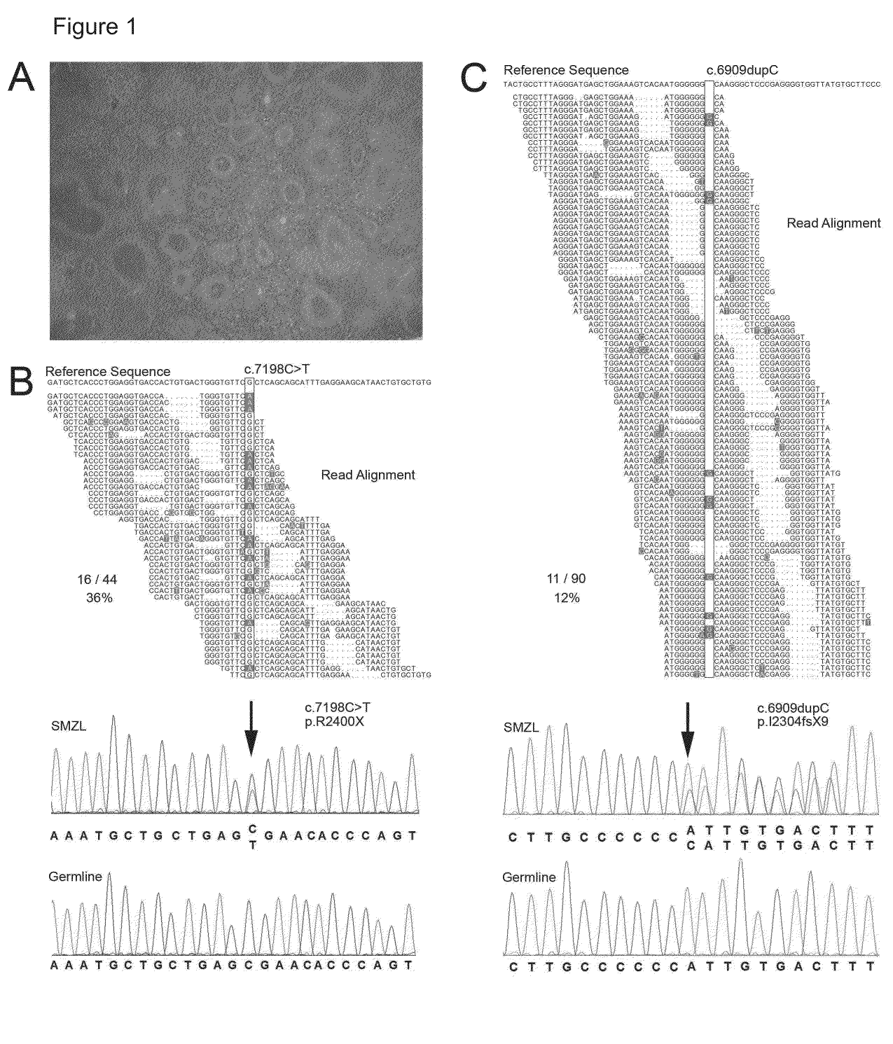

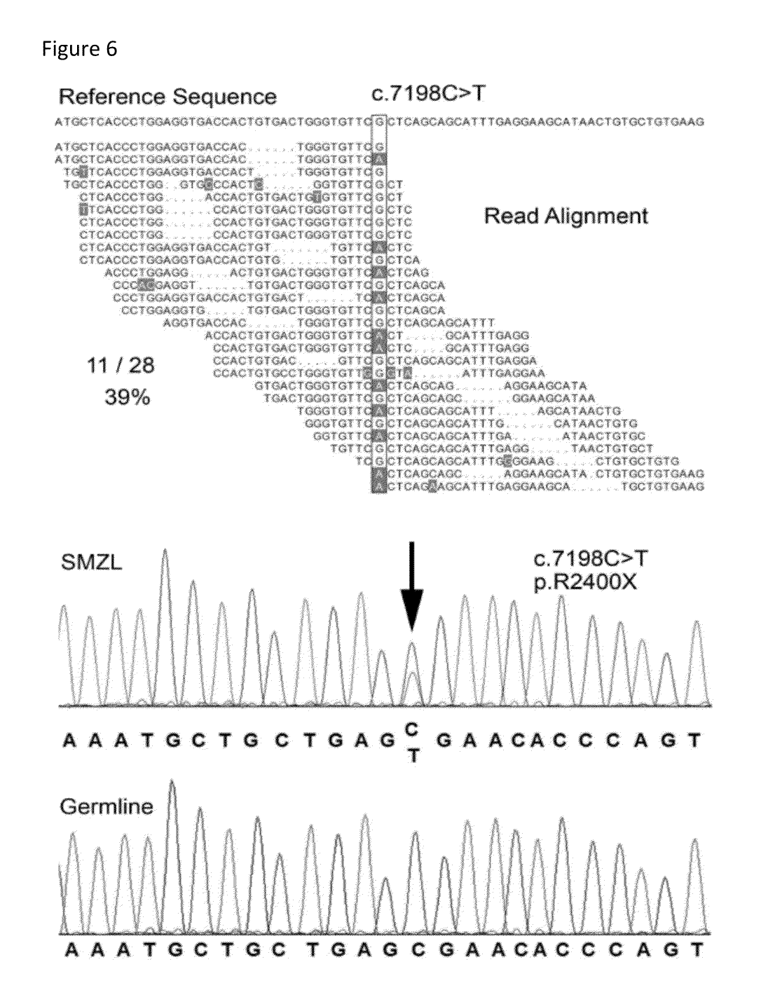

In total, 2,995 candidate genes were identified with at least one previously undocumented single nucleotide polymorphism (SNP) or small insertion/deletion event (indel) in at least one of the six SMZL genomes (comparison to dbSNP; not shown). Of these, 232 genes showed novel alterations in at least two of the six SMZL index genomes. These included mutations in epigenetic modifiers including MLL2 and MLL3 which have been previously reported to occur in follicular diffuse large B-cell lymphomas but not in marginal zone lymphomas (Morin et al., 2011 Nature. 476:298-303; Pasqualucci et al., 2011 Nat Genet. 43:830-837). In three of six index SMZL cases, variant call analysis identified NOTCH2 mutations predicted to lead to protein truncation in the distal C-terminal region in the transactivation (TAD) and proline/glutamate/serine/threonine-rich (PEST) domains. Two of these cases harbored the same p.R2400X nonsense amino acid substitution mutation and one case harbored a length-affecting mutation leading to a frameshift at residue p.I2304 (FIG. 1). These mutations result in deletion of known or predicted degradation motifs that regulate protein stability (Kopan and Ilagan, 2009 Cell 137:216-233). Moreover, NOTCH2 is known to regulate cell fate decisions during B-cell development influencing commitment to the marginal zone B-cell lineage (Saito et al., 2003 Immunity 18:675-685). Therefore, efforts were focused on further characterizing NOTCH2 mutations in SMZL as they are likely to be important to the pathogenesis of this disease. Using Sanger sequencing, the presence of these mutations in the index tumor samples (FIGS. 1 and 6; SMZL) and their somatic acquisition by testing matched constitutional tissues (Germline) was confirmed.

Prevalence of NOTCH2 Mutations in SMZL

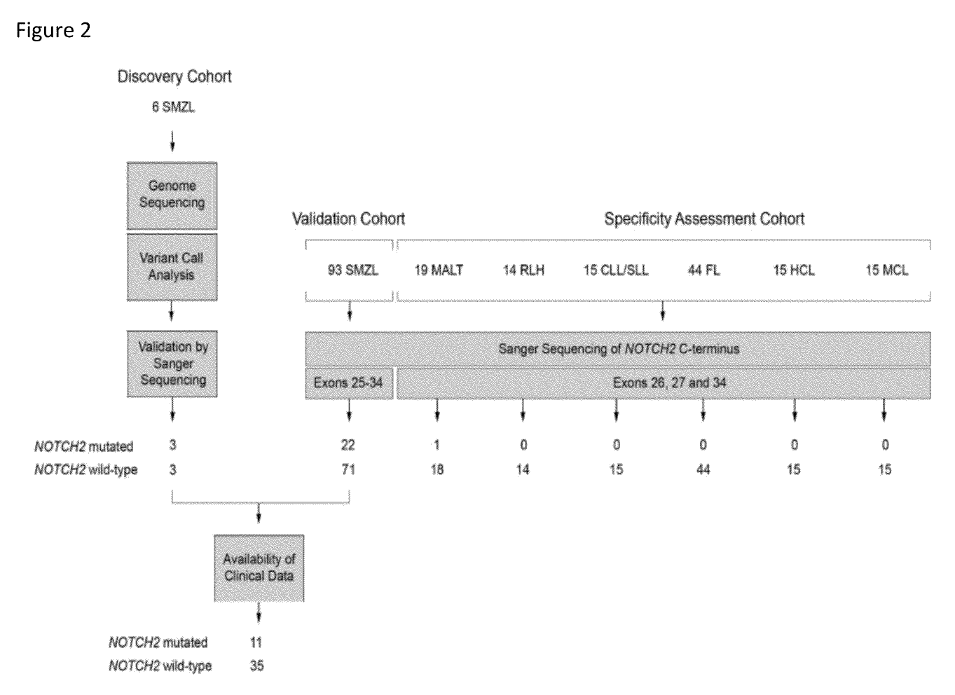

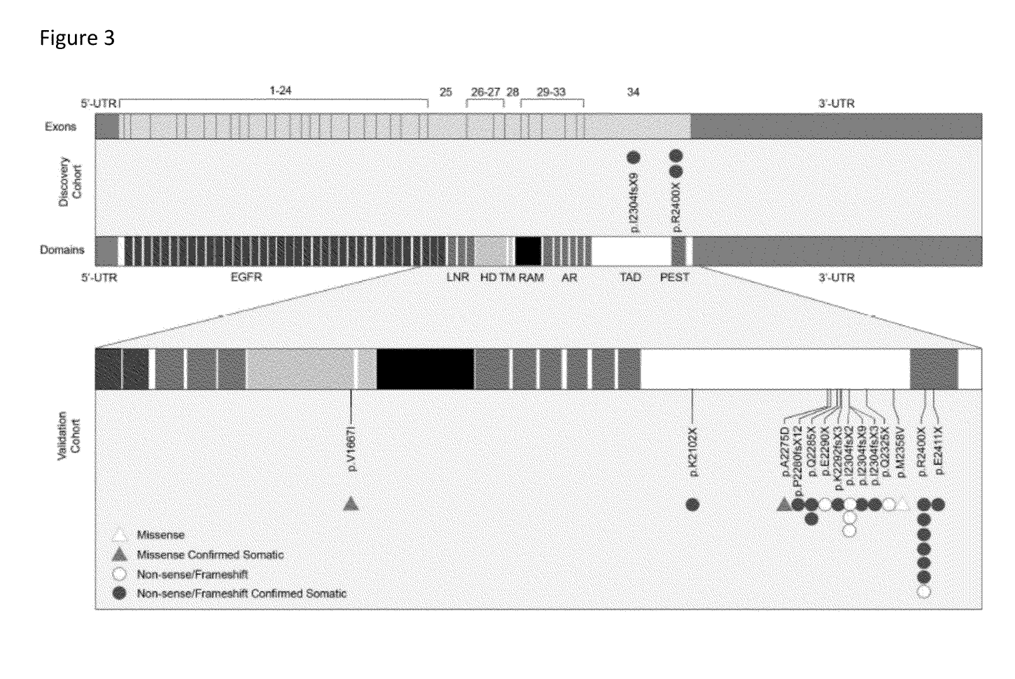

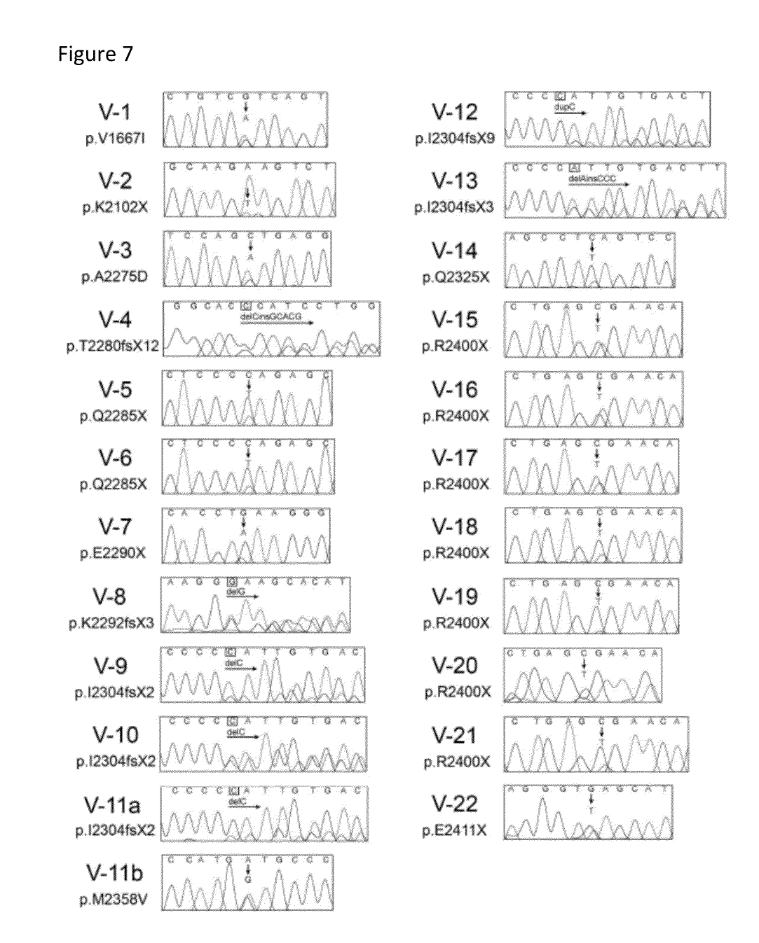

In order to establish the prevalence of NOTCH2 mutations among a larger SMZL cohort, targeted Sanger sequencing of exons 25 through 34 (FIG. 2) comprising all domains known to be important for intracellular NOTCH-family signaling was performed. These exons comprise three Lin-12-NOTCH repeat (LNR) domains (prevents ligand-independent activation), the HD (regulates ligand-independent activation), a single-pass trans-membrane region, RBP-J kappa-associated module (RAM) domain (required for NOTCH signaling), six ankyrin repeats (binds the CBF1/RBP-J kappa/suppressor of hairless/LAG-1 (CSL) transcription factor and Mastermind), the TAD, and the PEST domain important for regulating degradation of the NOTCH2 intracellular domain (NICD2) (FIG. 3). In total, 93 additional SMZL cases were screened by Sanger sequencing for mutations in the C-terminal of NOTCH2. A total of 11 novel mutations as well as seven additional p.R2400X and five additional frameshift mutations affecting the p.I2304 residue were discovered in these SMZL cases (FIGS. 3 and 7 and Table 2).

These mutations were largely length-affecting mutations (either frameshift or non-sense mutations) confined to the distal TAD and PEST domains and are predicted to cause truncation of the NOTCH2 protein, eliminating degradation signals in the PEST domain, thereby increasing the stability of the NICD2. A single missense mutation (p.V1667I) located in the HD is predicted to be equivalent to the p.V1722INOTCH1 mutation in T-ALL associated with ligand-independent NOTCH1 activation (Gordon et al., 2007 Nat Struct Mol Biol 14:295-300; Malecki et al., 2006 Mol Cell Biol 26:4642-4651). Overall, 25 of 99 SMZL cases (25.3%) harbored NOTCH2 mutations. Whereas most of these mutations were single heterozygous mutations, one of 25 SMZL patients had two distinct NOTCH2 mutations including both a length affecting mutation (p.I2304fsX2) and a missense variant (p.M2358V;

although constitutional tissue was not available to assess somatic acquisition). Of the 25 cases with NOTCH2 mutations, 19 patients had corresponding matched normal tissue. None of the constitutional tissues harbored sequence variants indicating somatic acquisition of NOTCH2 mutations detected in tumor tissue.

Having established a high frequency of NOTCH2 mutations in the validation cohort, the initial genomic sequencing screening data was queried for the existence of structural alterations affecting other genes in the NOTCH signaling pathway. This investigation identified predicted protein coding alterations affecting MAML2, a cofactor of the NOTCH2 transcriptional complex, in the three genomes that did not have NOTCH2 mutations. These alterations included previously reported p.Q237R and p.V836I variants as well as a novel p.G25W mutation. Sanger sequencing confirmed the variants in the corresponding tumor samples. However, the previously reported variants were present in corresponding germline tissue and thus were not somatically acquired. The novel p.G25W mutation was confirmed to be somatically acquired by direct Sanger sequencing. The mutation affects an amino acid with the N-terminal region of the MAML2 protein known to mediate protein-protein interactions with NOTCH family members. The prevalence of additional MAML2 mutations in the validation cohort were investigated. This identified a single additional somatic mutations in MAML2 (p.A11S) in a genome without an identified NOTCH2 mutation. Overall, the prevalence of putative impactful somatic mutations in MAML2 was therefore two out of 99 case (2.0%). No mutations were found in Fbw7 or other NOTCH-pathway related genes in the discovery cohort.

### Assessment of Functional Effect of NOTCH2 Mutations

Of the mutations identified in SMZL including the most frequently recurrent mutations at p.R2400 and p.I2304, most are predicted to prematurely truncate the protein prior to complete translation of the C-terminal PEST domain. These mutations are therefore predicted to abrogate the negative regulatory function of the PEST domain and lead to increased NICD2 stability with activation of downstream NOTCH2 signaling by gain-of-function. Additionally, the HD is known to protect from ligand-independent activation (Kojika and Griffin, 2001 Exp Hematol 29:1041-1052). The single missense mutation (p.V1667I) identified in the HD region would be predicted to disable this protection and thus trigger NOTCH2 intracellular signaling and promote downstream transcriptional activation.

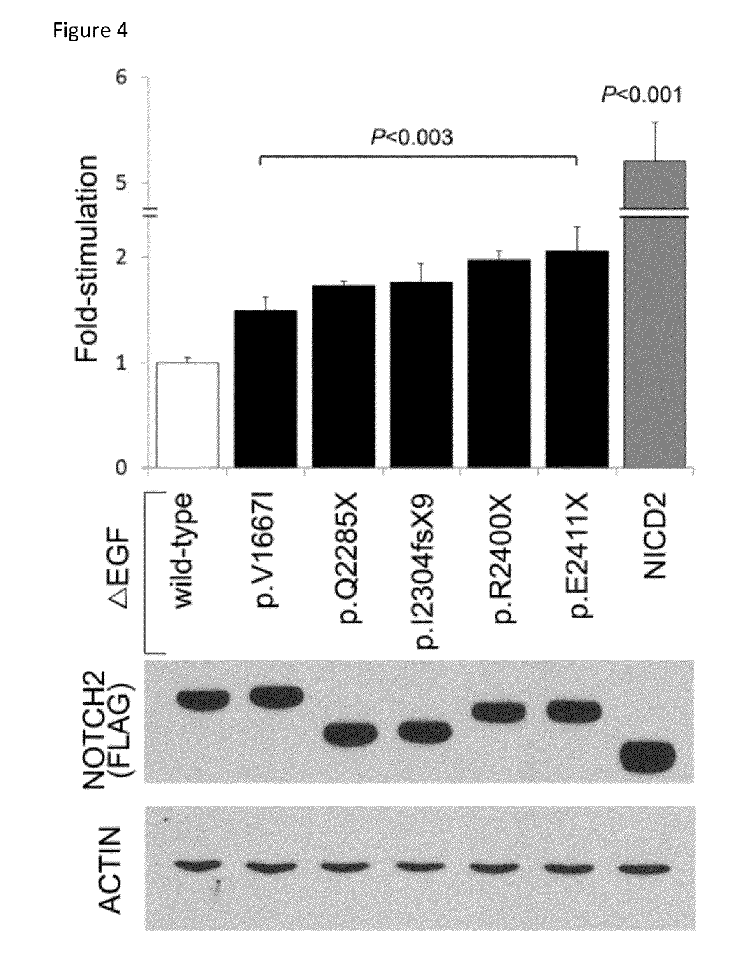

To test the functional effect of NOTCH2 mutations on NOTCH2 signaling, selected NOTCH2 mutant proteins (p.V1667I, p.Q2285X, p.I2304fsX9, p.R2400X and p.E2411X mutations) were transiently expressed into 293T cell lines and the effects on down-stream NOTCH2 signaling were assessed using a luciferase reporter system containing iterated CSL-binding sites derived from the HES 1 promoter (FIG. 4). All engineered mutant NOTCH2 proteins significantly induced the activity of the NOTCH2- responsive reporter gene when compared to wild-type NOTCH2 (P<0.003), indicating that the mutations lead to hyperactivation of NOTCH2 intracellular signaling.

### Specificity of NOTCH2 Mutations

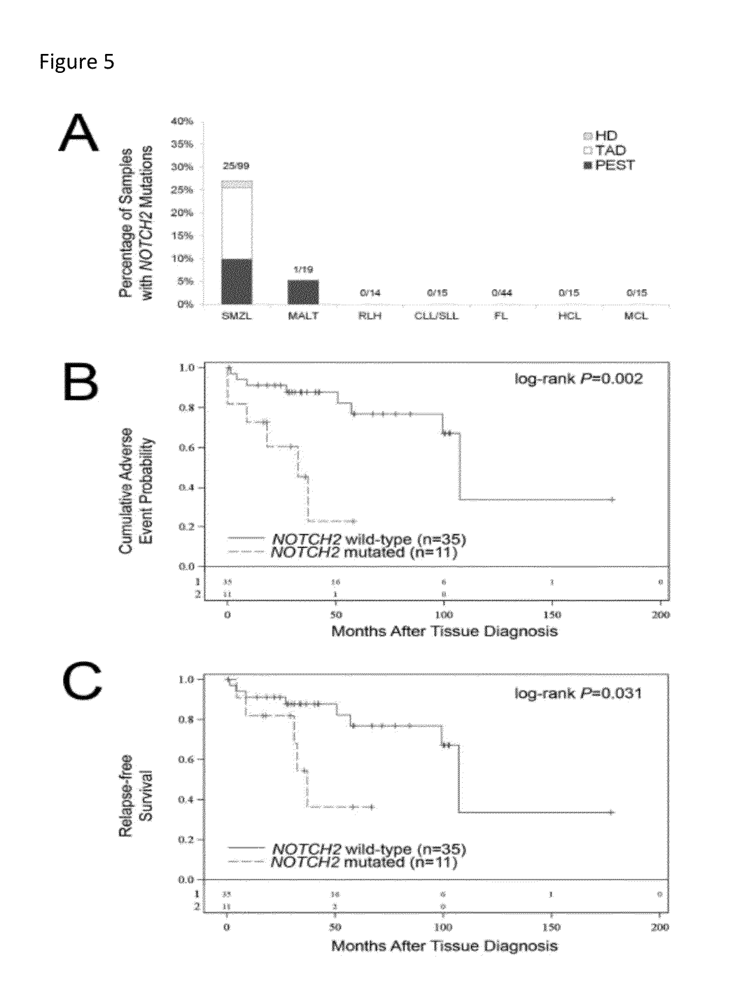

Having established the frequency of NOTCH2 mutations in SMZL, the specificity of these mutations for SMZL was assessed. Sanger sequencing was performed on CLL/SLL, FL, HCL, and MCL as well as RLH samples. No evidence of NOTCH2 mutations was identified in any of 103 cases of CLL/SLL, FL, HCL, MCL or RLH (FIGS. 2 and 5A). In addition to assessing 99 SMZL cases, 19 nodal and extranodal marginal zone lymphomas were assessed for the presence of NOTCH2 mutations and one sample (an extranodal marginal zone B-cell lymphoma of the breast) was identified that also harbored a heterozygous p.R2400X mutation (FIG. 5A). These data indicate a high frequency of NOTCH2 mutations in SMZLs and a lower (5.3%) frequency in non-splenic MZL. Taken together, these data indicate that activating mutations in NOTCH2 are specific to MZLs.

### Impact of NOTCH2 Mutations on Clinical Outcome

Having demonstrated the presence of NOTCH2 mutations in a subset of SMZL cases, it was determined whether the presence of these mutations influenced clinical outcomes. Time to adverse outcome, defined from tissue diagnosis to relapse, transformation or death was compared between patients harboring NOTCH2 mutations and those with wild-type

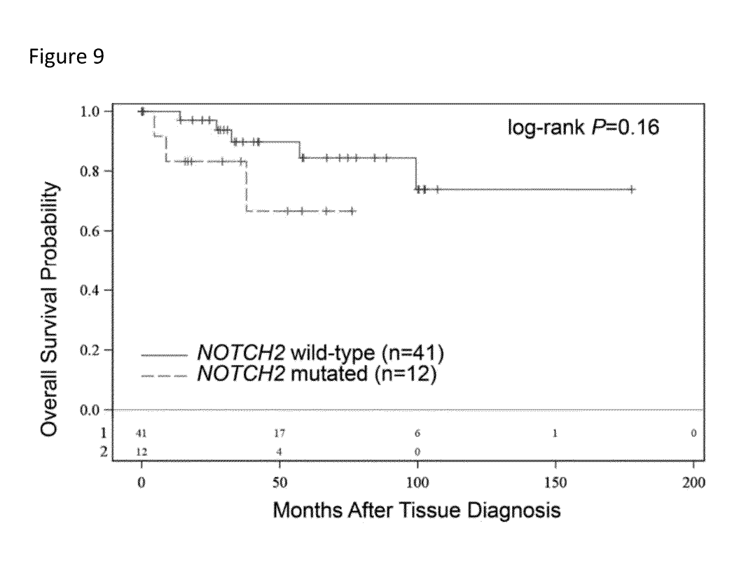

NOTCH2. Survival data was available for 46 patients from this study including 11 patients with NOTCH2 mutations and 35 patients with wild-type NOTCH2 with a median follow-up of 40 months (range: 0.7 to 177 months). Patients with NOTCH2 mutations had significantly shorter time to adverse outcome compared to patients with wild-type NOTCH2 (the median time to adverse outcome was 32.6 months in NOTCH2-mutated patients versus 107.2 months in patients without NOTCH2 mutations (P=0.002; FIG. 5B). After controlling for patient gender, performance status, age and stage at diagnosis, harboring NOTCH2 mutation is associated with shorter time to adverse outcome (Hazard Ratio=5.57; P=0.057). Furthermore, patients with NOTCH2 mutations also had significantly shorter relapse-free survival, defined from tissue diagnosis to relapse or death (P=0.031; FIG. 5C). In addition, there is a trend toward reduced overall survival (e.g., time to death) among patients with NOTCH2-mutated SMZL. However, this trend did not reach the level of statistical significance presumably due to a small sample size in this study (FIG. 9; P=0.16). Altogether, these results demonstrate that the presence of NOTCH2 mutation at diagnosis indicates worse patient outcome.

TABLE 1

| | | | | | Amino Acid | |
|---|---|---|---|---|---|---|
| | Forward Primer Sequence | Reverse Primer Sequence | Size | | Residues | |
| Fragment | for Amplification | for Amplification | (nt) | Exon | Begin | End |
| 34 | GTGGAGGTTTTCTAGAAACCTCA | GCACAATACTGGCTCAGACAG | 371 | 25 | 1336 | 1426 |
| 35 | GAGTCAGGCTGTGCCAGTA | CTGTTGCAGGCCTCATCACA | 312 | 25 | 1285 | 1430 |
| 36 | GGTAGCCGCTGTGAACTCTA | CGAGAAACTGAAGTGTGTTAGTGA | 351 | 25 | 1420 | 1504 |
| 37 | AGCTCCAGTCTAATCTGAGCTCT | CAGGTGGCATCAATACCACA | 208 | 26 | 1506 | 1545 |
| 38 | GGTGCAACAGTGAGGAGTGT | TAGCCTTGAAGTTCAGAAACCA | 328 | 26 | 1530 | 1620 |
| 39 | ATGACATGTTCTGCCTGACCT | CCTTTACACCAGTGCCACTC | 244 | 27 | 1821 | 1688 |
| 40 | ATCTAATGCTGACATTGAGAGGT | AGAGAGAGCCATGCTTACGCT | 192 | 28 | 1669 | 1706 |
| 41 | GTTGCTGTTGTCATCATTGTGT | AATCATGATTCAACAAGATATGC | 244 | 28 | 1680 | 1738 |
| 42 | GTGTCATGGTGGAAAGTGTTG | CAGATAATGGCTGACAATGGTG | 221 | 28 | 1799 | 1770 |
| 43 | AAACAATGGGAGATAAGCAGCGGTGGTG | GACAACAATGTGGAACCATG | 337 | 30 | 1771 | 1827 |
| 44 | CAAATAGAGCTGTTTCAACCATAG | ATTGGCATCTGCACCTGCATC | 310 | 31 | 1828 | 1865 |
| 45 | AGATGCAGAGGACTCTTCTGCT | TATTATTCAAGTGACTCTTCTCATGTT | 288 | 31 | 1870 | 1927 |
| 46 | CTACACTGTAGCCTCAGCTCTGAT | CCAAATCCCTGCCTTTCATC | 274 | 32 | 1928 | 1977 |
| 47 | CATTGTGCAAGTCATAGTGTCTT | GAATGGGCTTATAACTGAGGCA | 230 | 33 | 1977 | 2909 |
| 48 | CTCAAGAGTGTTATTAACATGTGTTC | CTTCAGGCTGAGGAAAGATCTG | 306 | 34 | 2018 | 2086 |
| 49 | TCGCATGCACCATGACATTG | GGATAAAGTTACTGAACTCTCAGAC | 292 | 34 | 2980 | 2148 |
| 50 | TTGCCAAGGAGGCAAAGGATG | CTCACTGAGGGAAGCACAGT | 310 | 34 | 2130 | 2210 |
| 51 | TGGGATCTTACAGGCCTCAC | CCAGGACCATACCAAACATC | 290 | 34 | 2185 | 2260 |
| 52 | CGCATGGAGGTGAATGAGA | CCATTTCTGGAATCTGGTACAT | 271 | 34 | 2280 | 2335 |
| 53 | CTAAAGGCAGTATTGCCCAAC | CTGGAGGTGACCACTGTGAC | 290 | 34 | 2320 | 2400 |
| 54 | GGCAGGTAGCTCAGACCAT | GTCAGGAGACTCTGGGGAT | 182 | 34 | 2365 | 2415 |
| 55 | GCTGAGCGAACACCCAGT | TGTTCCTCAGCAGCATTTACA | 283 | 34 | 2410 | 2471 |

TABLE 2

| | Forward Primer Sequence | Reverse Primer Sequence |
|---|---|---|
| Fragment | for Sequencing | for Sequencing |
| 34 | AGGTTTTCTAGAAACCTCAAACT | AATACTGGCTCAGACAGGTGG |
| 35 | CAGGCTGTGCCAGTAGCCC | TGCAGGCCTCATCACAGACG |
| 36 | GCCGCTGTGAACTCTACACG | AAACTGAAGTGTGTTAGTGACAGT |
| 37 | CCAGTCTAATCTGAGCTCTTTTG | TGGCATCAATACCACAATAA |
| 38 | CAACAGTGAGGAGTGTGGTT | CTTGAAGTTCAGAAACCAAACA |
| 39 | CATGTTCTGCCTGACCTGCAC | CCTTTACACCAGTGCCACTC |
| 40 | AATGCTGACATTGAGAGGTTAAT | AGAGCCATGCTTACGCTTTCG |
| 41 | CTGTTGTCATCATTCTGTTTAT | ATGATTCAACAAGATATGCTTTT |
| 42 | CATGGTGGAAAGTGTTGAAAA | TAATGGCTGACAATGGTGGTTC |
| 43 | AATGGGAGATAAGCAGCGGTGGTGGAGGCTC | ACAATGTGGAACCATGGGCA |
| 44 | TAGAGCTGTTTCAACCATAGGGTT | GCATCTGCACCTGCATCCAGG |
| 45 | GCAGAGGACTCTTCTGCTAACA | ATTCAAGTGACTCTTCTCATGTTCTTTACC |
| 46 | ACTGTAGCCTCAGCTCTGATGCCC | ATCCCTGCCTTTCATCCCTA |
| 47 | GTGCAAGTCATAGTGTCTTATAC | GGGCTTATAACTGAGGCACTGC |
| 48 | AGAGTGTTATTAACATGTGTTCTGTG | AGGCTGAGGAAAGATCTGTTGG |
| 49 | ATGCACCATGACATTGTGCG | AAAGTTACTGAACTCTCAGACAGTT |
| 50 | CAAGGAGGCAAAGGATGCCAA | CTGAGGGAAGCACAGTGCTG |
| 51 | ATCTTACAGGCCTCACCCAA | GACCATACCAAACATCTCAT |
| 52 | ATGGAGGTGAATGAGACCC | CCATTTCTGGAATCTGGTACAT |
| 53 | AGGCAGTATTGCCCAACCAGC | AGGTGACCACTGTGACTGGG |
| 54 | GGTAGCTCAGACCATTCTC | GGAGACTCTGGGGATGGTG |
| 55 | AGCGAACACCCAGTCACA | CCTCAGCAGCATTTACAAAAG |

TABLE 3

| | | | First Mutation | | Second Var [symbol] tion | | | Confirmed |
|---|---|---|---|---|---|---|---|---|
| Cohort | Diease | Identifer | Gene | Protein | Gene | Protein | Consequnce | Somatic |
| Discovery | SM [symbol] L | D-1 | [symbol] C | p. [symbol] X9 | | | [symbol] | Yes |
| Discovery | SM [symbol] L | D-2 | [symbol] T | p. [symbol] X | | | [symbol] | Yes |
| Discovery | SM [symbol] L | D-3 | [symbol] T | p. [symbol] X | | | [symbol] | Yes |
| Validation | SM [symbol] L | V-1 | [symbol] A | p. [symbol] | | | [symbol] | Yes |
| Validation | SM [symbol] L | V-2 | [symbol] T | p. [symbol] X | | | [symbol] | Yes |
| Validation | SM [symbol] L | V-3 | [symbol] A | p. [symbol] 2275D | | | [symbol] | Yes |
| Validation | SM [symbol] L | V-4 | [symbol] GCACG | p. [symbol] X12 | | | [symbol] | Yes |
| Validation | SM [symbol] L | V-5 | [symbol] T | p. [symbol] 2285X | | | [symbol] | Yes |
| Validation | SM [symbol] L | V-6 | [symbol] T | p. [symbol] 2285X | | | [symbol] | Yes |
| Validation | SM [symbol] L | V-7 | [symbol] A | p.E2299X | | | [symbol] | N/A |
| Validation | SM [symbol] L | V-8 | [symbol] G | p. [symbol] X3 | | | [symbol] | Yes |
| Validation | SM [symbol] L | V-9 | [symbol] C | p. [symbol] | | | [symbol] | N/A |
| Validation | SM [symbol] L | V-10 | [symbol] C | p. [symbol] X2 | | | [symbol] | N/A |
| Validation | SM [symbol] L | V-11 | [symbol] C | p. [symbol] X2 | [symbol] | [symbol] | [symbol] | N/A |
| Validation | SM [symbol] L | V-12 | [symbol] C | p. [symbol] X9 | | | [symbol] | Yes |
| Validation | SM [symbol] L | V-13 | [symbol] CCC | p. [symbol] X3 | | | [symbol] | Yes |
| Validation | SM [symbol] L | V-14 | [symbol] T | p.Q2325X | | | [symbol] | N/A |
| Validation | SM [symbol] L | V-15 | [symbol] T | p.R24 [symbol] X | | | [symbol] | Yes |
| Validation | SM [symbol] L | V-16 | [symbol] T | p.R24 [symbol] X | | | [symbol] | Yes |
| Validation | SM [symbol] L | V-17 | [symbol] T | p.R24 [symbol] X | | | [symbol] | Yes |
| Validation | SM [symbol] L | V-18 | [symbol] T | p. [symbol] X | | | [symbol] | Yes |
| Validation | SM [symbol] L | V-19 | [symbol] T | p.R24 [symbol] X | | | [symbol] | Yes |
| Validation | SM [symbol] L | V-20 | [symbol] T | p.R24 [symbol] X | | | [symbol] | Yes |
| Validation | SM [symbol] L | V-21 | [symbol] T | p.R24 [symbol] X | | | [symbol] | N/A |
| Validation | SM [symbol] L | V-22 | [symbol] T | p..E24 [symbol] | | | [symbol] | Yes |
| Specificity | MALT | S-1 | [symbol] T | p.R24 [symbol] X | | | [symbol] | N/A |

[symbol]

indicates data missing or illegible when filed

TABLE 4

| | Total | | | Positive | | | Nega [symbol] | | | |
|---|---|---|---|---|---|---|---|---|---|---|
| | avg | stdev | n | svg | stdev | n | avg | stdev | n | t-test P |
| Percent Male | 35% | | 71 | 22% | | 18 | 40% | | 53 | 0.19 |
| Age at Diagnosis | [symbol] | 12 | 71 | 63 | 9 | [symbol] | 61 | 13 | 53 | 0.63 |
| Age at Spl [symbol] tomy | 63 | 12 | 71 | 65 | 10 | 18 | 63 | 12 | 5 [symbol] | 0.62 |
| Stage at Diagnosis | 3.7 | 0.8 | 56 | 3.5 | 1.1 | 13 | 3.8 | 0.7 | 43 | |
| [symbol] | 2.4 | 0. [symbol] | 43 | [symbol] | 1.0 | [symbol] | 2.5 | [symbol] | 34 | [symbol] |
| Hgb, g/dL | 11.8 | 2.0 | 51 | 11.7 | 1.7 | 11 | 11.9 | 2.1 | 40 | 0.77 |
| LDH, U/L | [symbol] | 154 | 42 | 321 | 122 | [symbol] | 330 | 162 | 34 | 0.88 |
| Albumin, g/dL | 4.2 | 0.5 | 19 | 4.4 | 0.4 | 4 | 4.2 | 0.5 | 15 | [symbol] |
| WBC, [symbol] | [symbol] | 23.9 | 21 | 11.2 | 8.8 | 5 | 21.0 | 26.9 | [symbol] | 0.44 |
| P [symbol] | 2 [symbol] 1 | 109 | 19 | 160 | 5 [symbol] | 4 | 213 | 119 | 15 | 0.41 |
| [symbol] mg/L | 3.5 | 1.5 | [symbol] | 3.5 | 1.9 | 4 | 3.9 | 1.4 | 15 | 0.68 |

[symbol]

indicates data missing or illegible when filed

TABLE 5

| Genome | LeftChr | LeftPosition | LeftStand | Left gene | RightChr | RightPosition | RightStand |
|---|---|---|---|---|---|---|---|
| A01 | chr1 | 10,543,646 | + | PEX14 | chr1 | 10,546,089 | + |
| A01 | chr1 | 78,833,897 | + | [symbol] | chr1 | 78,835,838 | + |
| A01 | chr1 | 246, [symbol] ,887 | + | [symbol] MYD3 | chr1 | 246,3 [symbol] ,776 | + |
| A01 | chr2 | 41,913,661 | + | | chr6 | 13,191,446 | − |
| A01 | chr2 | 51,74 [symbol] 54 | − | | chr6 | 117,811,873 | − |
| A01 | chr2 | 51,74 [symbol] | [symbol] | | chr6 | 117,811,877 | [symbol] |
| A01 | chr2 | 55,634,1 [symbol] | + | CCDC [symbol] A | chr2 | 55,636,191 | + |
| A01 | chr2 | 77,657,766 | + | L [symbol] RTM4 | chr2 | 77, [symbol] 139 | + |
| A01 | chr2 | 144,011,246 | + | A [symbol] P15 | chr [symbol] | 154,152, [symbol] | − |
| A01 | chr2 | 175,507,361 | [symbol] | W [symbol] PF1 | chr2 | 175,509,424 | [symbol] |
| A01 | chr3 | 9, [symbol] | + | MTMR14 | chr3 | 9,697, [symbol] | + |
| A01 | chr3 | 30, [symbol] 778 | + | GADL1 | chr3 | 30,866,269 | + |
| A01 | chr3 | 120,1 [symbol] 4 | + | F [symbol] T [symbol] | chr3 | 120,1 [symbol] 951 | + |
| A01 | chr3 | 123,13 [symbol] | + | ADCY5 | chr3 | 123,136,2 [symbol] | + |
| A01 | chr3 | 152, [symbol] 79,998 | − | | chrX | 76,982,473 | − |
| A01 | chr3 | 172,715, [symbol] 21 | − | SPATA15 | chr3 | 173,1 [symbol] ,709 | + |
| A01 | chr4 | 10,554,912 | + | CLNK | chr4 | 10,552,747 | + |
| A01 | chr4 | 17,982,341 | + | L [symbol] RL | chr4 | 17,983,912 | + |
| A01 | chr4 | 83,636, [symbol] | + | SC [symbol] 5 | chr4 | 83,641,27 [symbol] | + |
| A01 | chr4 | 113,554,7 [symbol] | − | LAR [symbol] 7 | chr4 | 113,57 [symbol] | − |
| A01 | chr4 | 144,298,455 | + | GA [symbol] 1 | chr4 | 144,229,5 [symbol] | + |
| A01 | chr4 | 144, [symbol] | + | GA [symbol] 1 | chr4 | 144, [symbol] | + |
| A01 | chr5 | 16, [symbol] | + | MYO1 [symbol] | chr5 | 16, [symbol] | + |
| A01 | chr5 | 80,3 [symbol] | + | RASG [symbol] F2 | chr5 | 80,3 [symbol] | + |
| A01 | chr5 | 141,999,4 [symbol] 6 | + | FGF1 | chr5 | 141,999,9 [symbol] 2 | + |
| A01 | chr6 | 8 [symbol] ,993,7 [symbol] | + | BCKDHB | chr6 | 80,999,802 | + |
| A01 | chr7 | 55,91 [symbol] ,75 [symbol] | + | 4 [symbol] | chr7 | 55,917,452 | + |
| A01 | chr7 | 1 [symbol] 41 [symbol] 0 | + | ZAN | chr7 | 100,3 [symbol] | + |
| A01 | chr7 | 117,455,132 | + | CTTNBP2 | chr7 | 117,459 [symbol] | + |
| A01 | chr7 | 134,919,3 [symbol] | + | STRA [symbol] | chr7 | 134,920,901 | + |
| A01 | chr7 | 140,231,450 | + | DENND2A | chr7 | 140,231,914 | + |
| A01 | chr7 | 157,671,001 | + | PTPRN2 | chr7 | 157,671,947 | + |
| A01 | chr8 | 124,958,894 | + | FER1L [symbol] | chr8 | 124,961,978 | + |
| A01 | chr9 | 642,168 | + | KANK1 | chr9 | 648,239 | + |
| A01 | chr9 | 119,513,452 | [symbol] | A [symbol] TN2 | chr9 | 119,515,996 | − |
| A01 | chr9 | 12 [symbol] ,616,99 [symbol] | + | DENND1A | chr9 | 126,617,683 | + |
| A01 | chr10 | 68,169,701 | + | CTNNA3 | chr10 | 6 [symbol] ,217,728 | + |
| A01 | chr10 | 76, [symbol] ,531 | + | [symbol] AM [symbol] | chr10 | 7 [symbol] 52 | + |
| A01 | chr11 | 19,07 [symbol] | + | MRGPRX2 | chr11 | 19,0 [symbol] 0,395 | + |
| A01 | chr11 | 19,079,350 | [symbol] | MRGPRX2 | chr11 | 19,080,724 | − |
| A01 | chr11 | 65,933,634 | + | PAC [symbol] 1 | chr11 | 65,939, [symbol] 63 | + |
| A01 | chr11 | 66,657,990 | + | PC | chr11 | 66, [symbol] 5 [symbol] 04 | + |
| A01 | chr11 | 71,6 [symbol] ,390 | + | RNF121 | chr11 | 71, [symbol] 60,973 | + |
| A01 | chr12 | 44,691, [symbol] 32 | + | TMEM117 | chr12 | 44,692,914 | + |
| A01 | chr12 | 53,595,999 | + | ITGB7 | chr12 | 53,596,600 | + |
| A01 | chr12 | 86,695,694 | + | MGAT4C | chr12 | 8 [symbol] ,703, [symbol] 66 | + |
| A01 | chr12 | 129,787,758 | + | TMEM132D | chr12 | 129,78 [symbol] ,178 | + |
| A01 | chr13 | 2 [symbol] ,024,956 | + | ATP6A2 | chr13 | 2 [symbol] ,026,278 | + |
| A01 | chr13 | 93,363,4 [symbol] 6 | + | GPC5 | chr13 | 93,364,9 [symbol] 5 | + |
| A01 | chr14 | 33,603,6 [symbol] 8 | + | NPA93 | chr14 | 33,632,183 | + |
| A01 | chr14 | 79,159,147 | + | NRXN3 | chr11 | 79,165,647 | + |
| A01 | chr16 | 131,6 [symbol] 0 | + | MPG | chr16 | 132,250 | + |
| A01 | chr16 | 81,407,483 | − | GAN | chr16 | 81,408, [symbol] 94 | \hat{ } |
| A01 | chr17 | 2,39 [symbol] ,975 | + | METTL16 | chr17 | 2,402,799 | + |
| A01 | chr17 | 33,661,061 | + | [symbol] LFN11 | chr17 | 33,689,757 | + |
| A01 | chr17 | 33,667,3 [symbol] 2 | + | [symbol] LFN11 | chr17 | 33,6 [symbol] ,759 | + |
| A01 | chr17 | 33, [symbol] 47 | + | [symbol] LFN11 | chr17 | 33,700,494 | + |
| A01 | chr17 | 73,052,506 | \check{ } | KCTD2 | chr17 | 73,054,209 | \hat{ } |
| A01 | chr19 | 17,794,376 | + | UNC13A | chr19 | 17,794,814 | + |
| A01 | chr19 | 23,856,633 | + | ZNF675 | chr19 | 23, [symbol] 66,242 | + |
| A01 | chr19 | 53,477,443 | + | ZNF702P | chr19 | 53,477, [symbol] 62 | + |
| A01 | chr22 | 4 [symbol] ,074,72 [symbol] | + | FAM1 [symbol] A5 | chr22 | 49,075,600 | + |
| A01 | chrX | 2,351,84 [symbol] | [symbol] | DHR [symbol] X | chrX | 2,355,063 | [symbol] |
| A01 | chrX | 135, [symbol] 31,945 | + | MAP7D3 | chrX | 135,332,55 [symbol] | + |
| B01 | chr2 | 77,686,989 | [symbol] | LRRTM4 | chr2 | 77,693,012 | [symbol] |
| B01 | chr2 | 77,667,839 | + | LRRTM4 | chr2 | 77, [symbol] ,083 | + |
| B01 | chr2 | 143,926,139 | [symbol] | ARHGAP15 | chr2 | 143,927,714 | + |
| B01 | chr4 | 71,8 [symbol] 2,258 | + | MOBKL1A | chr4 | 71,804,751 | + |
| B01 | chr7 | 1 [symbol] 8, [symbol] 63,473 | − | [symbol] OPL | chr7 | 138,3 [symbol] ,051 | [symbol] |
| B01 | chr7 | 157,7 [symbol] ,125 | + | PTPRN2 | chr7 | 157,772,013 | − |
| B01 | chr9 | 21, [symbol] 42,066 | + | MTAP | chr9 | 21, [symbol] 54,133 | + |
| B01 | chr9 | 22,252,932 | − | | chr13 | [symbol] 7,7 [symbol] 3,729 | + |
| B01 | chr10 | 103,445,7 [symbol] 7 | + | FBXW4 | chr10 | 103,446,333 | + |
| B01 | chr11 | [symbol] 4,563,723 | [symbol] | DL [symbol] 2 | chr11 | 84,565,2 [symbol] 1 | [symbol] |
| B01 | chr11 | 84,563, [symbol] | + | DLG2 | chr11 | [symbol] 4,566,277 | + |
| B01 | chr11 | 94,080,417 | \check{ } | | chrX | 73,716,032 | \hat{ } |
| B01 | chr12 | 53,595,998 | + | ITGB7 | chr12 | 53,596,600 | + |
| B01 | chr12 | 70,679, [symbol] 5 | + | CNOT2 | chr12 | 70, [symbol] 0,974 | [symbol] |
| B01 | chr12 | 70,680,612 | [symbol] | CNOT2 | chr12 | 7 [symbol] ,682, [symbol] 55 | + |
| B01 | chr12 | 1 [symbol] 4,7 [symbol] ,691 | − | TXNRD1 | chr12 | 104,732,44 [symbol] | + |
| B01 | chr13 | 21,730, [symbol] 5 | + | SKA3 | chr13 | 21,731,227 | + |
| B01 | chr14 | 79,159,149 | + | NRXN3 | chr14 | 79,165,649 | + |
| B01 | chr17 | 9,749,659 | − | GLP2R | chr17 | 9,749,587 | + |
| B01 | chr17 | 33,681, [symbol] 81 | + | SLFN11 | chr17 | 33,689,757 | + |
| B01 | chr17 | 33,690, [symbol] 46 | − | SLFN11 | chr17 | 33,700,493 | + |
| B01 | chr18 | 38,626, [symbol] 45 | + | P [symbol] K3C3 | chr18 | 39,627,703 | + |
| B01 | chr19 | 17,359,2 [symbol] | + | | chr19 | 17,361, [symbol] 16 | + |
| B01 | chr19 | 37,922,139 | − | ZNF559 | chr19 | 3 [symbol] ,018,371 | + |
| B01 | chr21 | 22,852, [symbol] 29 | + | NCAM2 | chr21 | 22, [symbol] 2, [symbol] 0 | + |
| B01 | chr21 | 36,203,887 | + | RUNX1 | chr21 | 36,204, [symbol] 90 | + |
| B01 | chr22 | 17,2 [symbol] 7,5 [symbol] 7 | + | | chr22 | 17,273, [symbol] 77 | + |
| B01 | chr22 | 34,308, [symbol] 6 | − | LARGE | chr22 | 34,311,623 | + |
| B01 | chr22 | 34,309,375 | + | LARGE | chr22 | 34,309,999 | − |
| C01 | chr1 | 53,499, [symbol] 6 | − | [symbol] CP2 | chr1 | 53,499,667 | [symbol] |
| C01 | chr1 | 159,020,405 | + | [symbol] F [symbol] | chr1 | 159,021,049 | + |
| C01 | chr1 | 2 [symbol] ,351, [symbol] 93 | + | ARID [symbol] | chr1 | 235,355, [symbol] | + |
| C01 | chr2 | 135,4 [symbol] 1, [symbol] 52 | + | FM [symbol] 2 | chr2 | 153,493,458 | − |
| C01 | chr2 | 153,492,542 | − | FMNL2 | chr2 | 153,495,27 [symbol] | − |
| C01 | chr2 | 167,015,762 | [symbol] | | chr7 | [symbol] 5,637 | [symbol] |
| C01 | chr2 | 1 [symbol] 7,019,774 | − | | chr7 | 99,915,451 | + |
| C01 | chr4 | 152,732,426 | − | | chr5 | 121,670,726 | − |
| C01 | chr4 | 152,732, [symbol] | [symbol] | | chr5 | 121,670,7 [symbol] | [symbol] |
| C01 | chr5 | [symbol] ,885,142 | + | RGS7 [symbol] P | chr5 | 53, [symbol] 6,3 [symbol] 6 | + |
| C01 | chr5 | 129,480,450 | [symbol] | CH [symbol] Y3 | chr5 | 129,481,19 [symbol] | [symbol] |
| C01 | chr6 | 102,427,7 [symbol] | + | GRIK2 | chr6 | 102,42 [symbol] ,220 | [symbol] |
| C01 | chr7 | 4,252,922 | + | [symbol] DK1 | chr7 | 4,253, [symbol] 63 | [symbol] |
| C01 | chr7 | 110,121,3 [symbol] 1 | \hat{ } | | chr1 | 110,384,334 | \hat{ } |
| C01 | chr7 | 120,494,9936 | + | T [symbol] AN12 | chr7 | 1 [symbol] 0,4 [symbol] 6,0 [symbol] | − |
| C01 | chr9 | 15,231,259 | + | TTC39 [symbol] | chr9 | 15, [symbol] 71,978 | − |
| C01 | chr9 | 131,556, [symbol] 99 | + | T [symbol] C1D13 | chr9 | 131, [symbol] 7, [symbol] | + |
| C01 | chr10 | 56,445,9 [symbol] 2 | − | PCDH15 | chr10 | [symbol] 6,4 [symbol] | − |
| C01 | chr10 | [symbol] ,05 [symbol] ,480 | + | GRID1 | chr10 | 90,93 [symbol] | [symbol] |
| C01 | chr10 | 88,71 [symbol] ,882 | + | MMRN 2 | chr10 | [symbol] ,537,6 [symbol] | [symbol] |
| C01 | chr10 | 90,125,7 [symbol] 1 | + | RNL [symbol] | chr10 | 9 [symbol] ,77 [symbol] | [symbol] |
| C01 | chr10 | 177, [symbol] 1 | − | ATRNL1 | chr10 | 117, [symbol] 3 | − |
| C01 | chr11 | 34,172,14 [symbol] | [symbol] | | chr11 | 3 [symbol] ,174,1 [symbol] | [symbol] |
| C01 | chr11 | 121,9 [symbol] 2,273 | + | MIR10 [symbol] HG | chr11 | 122,722,674 | + |
| C01 | chr12 | 5 [symbol] ,595,998 | + | ITGB7 | chr12 | 5 [symbol] ,5 [symbol] ,600 | + |
| C01 | chr17 | 18,234,03 [symbol] | − | SHMT1 | chr17 | 18,2 [symbol] 4,3 [symbol] 9 | − |
| C01 | chr17 | 33,6 [symbol] 1,092 | + | [symbol] LFN [symbol] | chr17 | 33, [symbol] 89,757 | + |
| C01 | chr17 | 33,687,392 | + | [symbol] LFN [symbol] | chr17 | 33,689,759 | + |
| C01 | chr17 | 3 [symbol] ,690,846 | + | [symbol] LFN [symbol] | chr17 | 33,700,493 | + |
| C01 | chr17 | 4 [symbol] ,364, [symbol] | + | MAP3K14 | chr17 | 43,372,175 | + |
| C01 | chr18 | 77, [symbol] ,498 | [symbol] | ADNP2 | chr18 | 77,929, [symbol] 15 | [symbol] |
| C01 | chr22 | 37,415,327 | + | T [symbol] T | chr22 | 37,420,695 | + |
| D01 | chr1 | 172,2 [symbol] 2, [symbol] | + | DNM3 | chr1 | 172, [symbol] 1,753 | + |
| D01 | chr2 | 173,3 [symbol] 2,495 | + | ITGA [symbol] | chr2 | 173,3 [symbol] 4,472 | + |
| D01 | chr3 | 100,334,873 | [symbol] | GPR128 | chr3 | 100,44 [symbol] ,152 | [symbol] |
| D01 | chr3 | 123,135,519 | [symbol] | ADCY5 | chr3 | 123,136,265 | + |
| D01 | chr5 | 129,020,376 | + | ADAMT [symbol] 19 | chr5 | 129,024,195 | + |
| D01 | chr5 | 149,230,181 | − | PPARGC18 | chr5 | 149,270,199 | − |
| D01 | chr7 | 1 [symbol] 26, [symbol] 00 | + | HDAC9 | chr7 | 18,826,467 | + |
| D01 | chr8 | 57,048,719 | − | | chr8 | 57,09 [symbol] ,539 | − |
| D01 | chr9 | 11 [symbol] ,667 [symbol] | [symbol] | LPAR1 | chr9 | 113,669,264 | + |
| D01 | chr10 | 1 [symbol] 5,128,324 | + | TAF [symbol] | chr10 | 105,133,114 | + |
| D01 | chr14 | 47,672,012 | [symbol] | MDGA2 | chr14 | 47,679,230 | + |
| D01 | chr15 | 85,38 [symbol] ,985 | [symbol] | ALPK3 | chr15 | 85,381,400 | + |
| D01 | chr16 | 85,381,131 | [symbol] | ALPK3 | chr15 | [symbol] 5,381,398 | − |
| D01 | chr16 | 83,196,147 | [symbol] | CDG13 | chr16 | 83,209,726 | + |
| D01 | chr17 | 44,887,353 | + | WNT3 | chr17 | 44,887,685 | [symbol] |
| D01 | chr21 | 43,703,919 | + | ABCG1 | chr21 | 43,704, [symbol] 97 | + |
| D01 | chr22 | 4 [symbol] ,924,695 | + | CELSR1 | chr22 | 46,925,569 | + |
| E01 | chr1 | 162,378,221 | + | [symbol] H2D1 [symbol] | chr1 | 162,378,877 | + |
| E01 | chr2 | 32,201,5 [symbol] 2 | [symbol] | MEMO1 | chr2 | 32,203,192 | [symbol] |
| E01 | chr2 | 46,128,512 | + | PRKCE | chr2 | 46,132,406 | + |
| E01 | chr2 | 2 [symbol] ,24 [symbol] ,836 | [symbol] | PARD3B | chr2 | 206,255,783 | + |
| E01 | chr4 | 6,635,439 | [symbol] | | chr5 | 90,979,006 | + |
| E01 | chr4 | 6,635,748 | [symbol] | | chr6 | 90,987,997 | − |
| E01 | chr4 | 128,954,985 | − | | chr17 | 49,977,259 | − |
| E01 | chr5 | 9267,219 | + | SEMA5A | chr5 | 9,275,423 | + |
| E01 | chr6 | 99,979,0 [symbol] 6 | − | BACH2 | chr17 | 70,860,680 | − |
| E01 | chr7 | 133,039,961 | + | EXOC4 | chr7 | 133,040,5 [symbol] | + |
| E01 | chr7 | 151,552,174 | + | PRKAG2 | chr7 | 151,552,718 | + |
| E01 | chr9 | 131,556,898 | + | TBC1D13 | chr9 | 131,557,883 | + |
| E01 | chr10 | 123,827,180 | [symbol] | TCC2 | chr10 | 123,831,512 | + |
| E01 | chr12 | 18,222,218 | + | | chr12 | 18,234,13 [symbol] | − |
| E01 | chr12 | 18,222,227 | [symbol] | | chr12 | 18,234,192 | + |
| E01 | chr12 | [symbol] 1,376,811 | + | | chr12 | [symbol] 1,380,334 | + |
| E01 | chr12 | 53,595,988 | [symbol] | ITGB7 | chr12 | 53, [symbol] 96,590 | + |
| E01 | chr12 | 99,978,721 | [symbol] | ANK [symbol] 1 [symbol] | chr12 | 99,982,702 | + |
| E01 | chr16 | 4,067,093 | [symbol] | ADCY9 | chr16 | 4,067,573 | + |
| E01 | chr17 | 33,681,081 | | SLFN11 | chr17 | 33,689,757 | [symbol] |
| E01 | chr17 | 33,687,392 | + | SLFN11 | chr17 | 33,689,759 | + |
| E01 | chr17 | 33,690,847 | [symbol] | SLFN11 | chr17 | 33,700,494 | + |
| E01 | chr17 | 71,5 [symbol] 2,137 | − | SDK2 | chr17 | 71,5 [symbol] 2,986 | + |
| E01 | chr18 | 24,134,023 | [symbol] | KCTD1 | chr18 | 24,134,444 | [symbol] |
| E01 | chrX | 19,640,905 | [symbol] | SH3KBP1 | chrX | 19,641,54 [symbol] | − |
| E01 | chrX | 32,931,076 | [symbol] | DMD | chrX | 32,931,504 | + |
| F01 | chr1 | 162,777,028 | + | NPL | chr1 | 182,782,290 | + |
| F01 | chr2 | 2 [symbol] ,720,813 | [symbol] | PLB1 | chr2 | 28,721,355 | + |
| F01 | chr2 | 148,807,786 | [symbol] | M [symbol] D5 | chr2 | 148,813,752 | + |
| F01 | chr3 | 61,827,238 | + | PTPRG | chr3 | 61,837,175 | + |
| F01 | chr3 | 124,001,98 [symbol] | − | KALRN | chr18 | 75,652,754 | − |
| F01 | chr3 | 173,240,733 | [symbol] | NLGN1 | chr3 | 173,241,713 | + |
| F01 | chr4 | 2,941,530 | [symbol] | NOP14 | chr12 | 16,970,231 | [symbol] |
| F01 | chr4 | 2,941,851 | [symbol] | NOP14 | chr12 | 16,970,253 | [symbol] |
| F01 | chr4 | 21,469,843 | [symbol] | KCNIP4 | chr4 | 110,24 [symbol] ,224 | [symbol] |
| F01 | chr4 | 169,1 [symbol] 5, [symbol] 1 [symbol] | − | DDX60 | chr7 | 116,806, [symbol] 01 | [symbol] |
| F01 | chr4 | 1 [symbol] 9,013,485 | [symbol] | TRIML2 | chr4 | 1 [symbol] 9,015,126 | [symbol] |
| F01 | chr5 | 14,74 [symbol] ,624 | + | ANKH | chr5 | 14,750,271 | + |
| F01 | chr5 | 14,749,156 | [symbol] | ANKH | chr5 | 14,753,376 | + |
| F01 | chr5 | 14,749,156 | [symbol] | ANKH | chr5 | 14,753,376 | [symbol] |
| F01 | chr5 | 14,749,513 | + | ANKH | chr5 | 18,803,73 [symbol] | [symbol] |
| F01 | chr5 | 14,749,5 [symbol] 6 | − | ANKH | chr5 | 14,753,3 [symbol] 1 | − |
| F01 | chr5 | 14,75 [symbol] ,143 | [symbol] | ANKH | chr13 | 10 [symbol] ,513,997 | − |
| F01 | chr5 | 14,751,670 | − | ANKH | chr5 | 14,751,898 | + |
| F01 | chr5 | 14,753,888 | [symbol] | ANKH | chr5 | 14,753,922 | − |
| F01 | chr5 | 18,065,378 | [symbol] | | chr5 | 41,921,426 | |
| F01 | chr5 | 18,861,724 | − | | chr5 | 41,792,602 | − |
| F01 | chr5 | 18,888,418 | [symbol] | | chr5 | 41,071,123 | + |
| F01 | chr5 | 28,352,392 | [symbol] | | chr5 | 41,343,636 | + |
| F01 | chr5 | 28,3 [symbol] 4,401 | + | | chr5 | 41,831,861 | + |
| F01 | chr5 | 26,649,138 | [symbol] | | chr5 | 41,831,486 | [symbol] |
| F01 | chr5 | 41,198,129 | [symbol] | C6 | chr5 | 41,873,752 | + |
| F01 | chr5 | 41,334,175 | [symbol] | PLCXD3 | chr5 | 41,826,701 | + |
| F01 | chr5 | 41,805,1 [symbol] 1 | [symbol] | OXCT1 | chr5 | 41,862,874 | − |
| F01 | chr6 | 4,928,004 | + | CDYL | chr5 | 4,928,621 | + |
| F01 | chr7 | 4,229,796 | − | [symbol] DK1 | chr7 | 4,300,538 | + |
| F01 | chr7 | 103,2 [symbol] ,122 | [symbol] | RELN | chr7 | 103,288,134 | + |
| F01 | chr7 | 114,045, [symbol] 55 | + | ZNF555 | chr8 | 73,139,003 | + |
| F01 | chr8 | 97,792,198 | [symbol] | PGCP | chr8 | 97,792,592 | + |
| F01 | chr9 | 80,003,450 | [symbol] | VPS13A | chr9 | 80,007,848 | − |
| F01 | chr9 | 80,003,452 | \check{ } | VP [symbol] 13A | chr9 | 80,007,849 | [symbol] |
| F01 | chr9 | 113, [symbol] 7,456 | \hat{ } | LPAR1 | chr9 | 113, [symbol] 9,157 | [symbol] |
| F01 | chr9 | 113,667,553 | + | LPAR1 | chr9 | 113,669,264 | + |
| F01 | chr9 | 138,96 [symbol] ,180 | − | NACC2 | chr9 | 138,963,225 | − |
| F01 | chr10 | 58,789,805 | \check{ } | | chr15 | 60,713,666 | + |
| F01 | chr10 | 58,789,909 | − | | chr15 | 60,713,908 | [symbol] |
| F01 | chr10 | 8 [symbol] ,642,127 | + | BMPR1A | chr10 | 88,642,670 | + |
| F01 | chr11 | 108,026,918 | [symbol] | | chr11 | 108,122,646 | + |
| F01 | chr12 | 32,330,618 | [symbol] | BICD1 | chr12 | 32,335,819 | [symbol] |
| F01 | chr13 | 44,958,987 | + | [symbol] ERP2 | chr13 | 106,470,584 | + |
| F01 | chr13 | 52,719,211 | \hat{ } | NEK3 | chr13 | 107,935,6674 | [symbol] |
| F01 | chr13 | 93,965,435 | − | GPC6 | chr13 | 112,524,234 | − |
| F01 | chr15 | 40,102,355 | + | GPR175 | chr15 | 40,104,131 | + |
| F01 | chr17 | 5,270,843 | [symbol] | RABEP1 | chr17 | 5,271,336 | [symbol] |
| F01 | chr17 | 31,632,861 | + | ACCN1 | chr17 | 31,636,171 | + |
| F01 | chr17 | 44,887,353 | − | WNT3 | chr17 | 44,887,686 | [symbol] |
| F01 | chr17 | 4 [symbol] ,400,405 | + | SKAP1 | chr17 | 4 [symbol] ,402,558 | + |
| F01 | chr18 | 9,284,974 | [symbol] | ANKRD12 | chr18 | 9,286,141 | [symbol] |
| F01 | chr19 | 4,291,820 | + | | chr19 | 4,292,423 | + |
| F01 | chr19 | 53,447,404 | + | ZNF702P | chr19 | 53,477,955 | + |
| F01 | chrX | 17,061,042 | − | REP [symbol] 2 | chrX | 17,063,314 | [symbol] |
| F01 | chrX | 19,860,65 [symbol] | + | SH3KBP1 | chrX | 19,861,144 | − |
| F01 | chrX | 19,860,751 | \hat{ } | SH3KBP1 | chrX | 19,861,300 | [symbol] |

| | Right gene | [symbol] nterchromosome | StandConsist [symbol] | Distance | Displayed |
|---|---|---|---|---|---|
| A01 | PEX14 | N | Y | 2,443 | |
| A01 | | N | Y | 1.941 | |
| A01 | [symbol] MYD3 | N | Y | 19,889 | |
| A01 | PHACTR1 | Y | N | | yes |
| A01 | DCBLD1 | Y | Y | | yes |
| A01 | DCBLD1 | Y | Y | | yes |
| A01 | CCDC [symbol] A | N | Y | 2 [symbol] | |
| A01 | L [symbol] RTM4 | N | Y | 1 [symbol] 3 | |
| A01 | | Y | N | | yes |
| A01 | W [symbol] PF1 | N | Y | 1 [symbol] 3 | |
| A01 | MTMR14 | N | Y | 82 [symbol] | |
| A01 | GADL1 | N | Y | 491 | |
| A01 | F [symbol] T [symbol] | N | Y | 3,307 | |
| A01 | ADCY5 | N | Y | 746 | |
| A01 | ATRX | Y | Y | | yes |
| A01 | NLGN1 | N | N | 417,888 | |
| A01 | CLNK | N | Y | 7 [symbol] | |
| A01 | LCORL | N | Y | 1 [symbol] 571 | |
| A01 | SC [symbol] 5 | N | Y | 5.262 | |
| A01 | | N | Y | 14,127 | |
| A01 | GA [symbol] 1 | N | Y | 1,075 | |
| A01 | GA [symbol] 1 | N | Y | [symbol] 99 | |
| A01 | MYO1 [symbol] | N | Y | 1.02 [symbol] | |
| A01 | RASGRF2 | N | Y | 70 [symbol] | |
| A01 | FGF1 | N | Y | 57 [symbol] | |
| A01 | BCKDHB | N | Y | 6, [symbol] 96 | |
| A01 | 40 [symbol] | N | Y | 3, [symbol] | |
| A01 | ZAN | N | Y | 2.009 | |
| A01 | CTTNBP2 | N | Y | 4. [symbol] 06 | |
| A01 | STRA [symbol] | N | Y | 1,165 | |
| A01 | DENND2A | N | Y | 4 [symbol] 4 | |
| A01 | PTPRN2 | N | Y | 946 | |
| A01 | FER1L6 | N | Y | 3,0 [symbol] 4 | |
| A01 | KANK1 | N | Y | 6.071 | |
| A01 | ASTN2 | N | Y | 2,544 | |
| A01 | DENND1A | N | Y | 6 [symbol] 5 | |
| A01 | CTBBA3 | N | Y | 48,027 | |
| A01 | [symbol] AM [symbol] | N | Y | 7,121 | |
| A01 | MRGPRX2 | N | Y | 1, [symbol] 62 | |
| A01 | MRGPRX2 | N | Y | 1,374 | |
| A01 | PAC [symbol] | N | Y | 5,429 | |
| A01 | PC | N | Y | 914 | |
| A01 | RNF121 | N | Y | 563 | |
| A01 | TMEM117 | N | Y | 1,082 | |
| A01 | ITGB7 | N | Y | 6 [symbol] 2 | |
| A01 | MGAT4C | N | Y | 7.372 | |
| A01 | TMEM132D | N | Y | 420 | |
| A01 | ATP8A2 | N | Y | 1,322 | |
| A01 | GPC5 | N | Y | 1,489 | |
| A01 | NPA93 | N | Y | 28,615 | |
| A01 | NRXN3 | N | Y | 6,500 | |
| A01 | MPG | N | Y | 570 | |
| A01 | GAN | N | Y | 611 | |
| A01 | METTL16 | N | Y | 3, [symbol] 24 | |
| A01 | [symbol] LFN11 | N | Y | 8,676 | |
| A01 | [symbol] LFN11 | N | Y | 2,367 | |
| A01 | [symbol] LFN11 | N | Y | 9,647 | |
| A01 | KCTD2 | N | Y | 1,703 | |
| A01 | UNC13A | N | Y | 438 | |
| A01 | ZNF675 | N | Y | 9,609 | |
| A01 | ZNF702P | N | Y | 519 | |
| A01 | FAM19A5 | N | Y | 871 | |
| A01 | DHR [symbol] X | N | Y | 3,223 | |
| A01 | MAP7D3 | N | Y | 6 [symbol] 5 | |
| B01 | LRRTM4 | N | Y | 6,043 | |
| B01 | LRRTM4 | N | Y | 6,044 | |
| B01 | ARHGAP15 | N | Y | 1,575 | |
| B01 | MO [symbol] KL1A | N | Y | 2,493 | |
| B01 | | N | Y | 1,57 [symbol] | |
| B01 | PTPRN2 | N | Y | 2, [symbol] | |
| B01 | MTAP | N | Y | 12. [symbol] 47 | |
| B01 | PCDH9 | Y | N | | yes |
| B01 | F [symbol] XW4 | N | Y | 601 | |
| B01 | DL [symbol] 2 | N | Y | 1.5 [symbol] | |
| B01 | DLG2 | N | Y | 1,477 | |
| B01 | [symbol] LC16 [symbol] | Y | Y | | yes |
| B01 | ITGB7 | N | Y | 602 | |
| B01 | CNOT2 | N | N | 1, [symbol] 79 | |
| B01 | CNOT2 | N | N | 1,453 | |
| B01 | TXNRD1 | N | Y | 1,757 | |
| B01 | SKA3 | N | Y | 322 | |
| B01 | NRXN3 | N | Y | 6.5 [symbol] | |
| B01 | GLP2R | N | Y | 528 | |
| B01 | SLFN11 | N | Y | 8,676 | |
| B01 | SLFN11 | N | Y | 9,547 | |
| B01 | P [symbol] 3C3 | N | Y | 856 | |
| B01 | USHBP [symbol] | N | Y | 1,764 | |
| B01 | ZNF793 | N | Y | 96,232 | |
| B01 | NCAM2 | N | Y | 631 | |
| B01 | RUNX1 | N | Y | 1,003 | |
| B01 | [symbol] KR [symbol] | N | Y | 15,870 | |
| B01 | LARGE | N | N | 2,6 [symbol] 7 | |
| B01 | LARGE | N | N | 514 | |
| C01 | [symbol] CP2 | N | Y | 661 | |
| C01 | [symbol] F [symbol] | N | Y | 644 | |
| C01 | ARID4 [symbol] | N | Y | 3,427 | |
| C01 | FMNL2 | N | N | 1,6 [symbol] 6 | |
| C01 | FMNL2 | N | N | 2,73 [symbol] | |
| C01 | [symbol] UD31 | Y | N | | yes |
| C01 | BUD31 | Y | N | | yes |
| C01 | [symbol] NCAIP | Y | Y | | yes |
| C01 | [symbol] NCAIP | Y | Y | | yes |
| C01 | RG [symbol] 7 [symbol] P | N | Y | 1,174 | |
| C01 | CH [symbol] Y3 | N | Y | 743 | |
| C01 | GRIK2 | N | Y | 433 | |
| C01 | [symbol] DK1 | N | Y | 941 | |
| C01 | IMM2L | N | Y | [symbol] 62, [symbol] | |
| C01 | T [symbol] AN12 | N | Y | 1,0 [symbol] | |
| C01 | | N | Y | 140,709 | |
| C01 | T [symbol] C1D13 | N | Y | 9 [symbol] 5 | |
| C01 | PCDH15 | N | Y | 2 [symbol] | |
| C01 | | N | N | 2,887,853 | yes |
| C01 | ATAD1 | N | N | [symbol] 20,74 [symbol] | yes |
| C01 | | N | N | 5 [symbol] 5, [symbol] 15 | yes |
| C01 | ATRNL1 | N | Y | 4,112 | |
| C01 | A [symbol] T [symbol] 2 | N | Y | 1,958 | |
| C01 | CRTAM | N | Y | 760,4 [symbol] 1 | yes |
| C01 | ITGB7 | N | Y | 602 | |
| C01 | SHMT1 | N | Y | 339 | |
| C01 | [symbol] LFN [symbol] | N | Y | [symbol] 5 | |
| C01 | [symbol] LFN [symbol] | N | Y | 2,367 | |
| C01 | [symbol] LFN [symbol] | N | Y | 9,547 | |
| C01 | MAP3K14 | N | Y | 7,510 | |
| C01 | PARD [symbol] G | N | Y | 59,517 | |
| C01 | MP [symbol] T | N | Y | 5,368 | |
| D01 | DNM3 | N | Y | 9,149 | |
| D01 | ITGA [symbol] | N | Y | 1,977 | |
| D01 | TFG | N | Y | 111,279 | |
| D01 | ADCY5 | N | Y | 74 [symbol] | |
| D01 | ADAMT [symbol] 19 | N | Y | 3,819 | |
| D01 | PDE [symbol] A | N | Y | 40,016 | |
| D01 | HDAC9 | N | Y | 167 | |
| D01 | FLAG1 | N | Y | 49, [symbol] 2 [symbol] | |
| D01 | LPAR1 | N | Y | 1,711 | |
| D01 | TAF5 | N | Y | 4,790 | |
| D01 | MDGA2 | N | Y | 6,218 | |
| D01 | ALPK3 | N | N | 41 [symbol] | |
| D01 | ALPK3 | N | N | 267 | |
| D01 | CDH13 | N | Y | 13,579 | |
| D01 | WNT3 | N | Y | 3 [symbol] 2 | |
| D01 | ABCG1 | N | Y | 484 | |
| D01 | CELSR1 | N | Y | 874 | |
| E01 | [symbol] H2D1 [symbol] | N | Y | 656 | |
| E01 | MEMO1 | N | Y | 1,6 [symbol] | |
| E01 | PRKCE | N | Y | 3,894 | |
| E01 | PARD3B | N | Y | 8,947 | |
| E01 | BACH2 | Y | N | | yes |
| E01 | BACH2 | Y | N | | yes |
| E01 | CA10 | Y | Y | | yes |
| E01 | SEMA5A | N | Y | [symbol] ,204 | |
| E01 | SLC39A11 | Y | Y | | yes |
| E01 | EXOC4 | N | Y | 597 | |
| E01 | PRKAG2 | N | N | 544 | |
| E01 | TBC1D13 | N | Y | 985 | |
| E01 | TACC2 | N | Y | 4,332 | |
| E01 | RERGL | N | N | 11,920 | |
| E01 | RERGL | N | N | 11,965 | |
| E01 | SLC11A2 | N | Y | 3,523 | |
| E01 | ITGB7 | N | Y | 602 | |
| E01 | ANK [symbol] 1 [symbol] | N | Y | 3,981 | |
| E01 | ADCY9 | N | Y | 480 | |
| E01 | SLFN11 | N | Y | 8,676 | |
| E01 | SLFN11 | N | Y | 2,367 | |
| E01 | SLFN11 | N | Y | 9,647 | |
| E01 | SDK2 | N | N | 849 | |
| E01 | KCTD1 | N | Y | 421 | |
| E01 | SH3KBP1 | N | Y | 643 | |
| E01 | DMD | N | Y | 52 [symbol] | |
| F01 | NPL | N | Y | 5,262 | |
| F01 | PLB1 | N | Y | 542 | |
| F01 | MBD5 | N | Y | 5,97 [symbol] | |
| F01 | PTPRG | N | Y | 9,937 | |
| F01 | | Y | Y | | yes |
| F01 | NLGN1 | N | Y | 979 | |
| F01 | | Y | Y | | yes |
| F01 | | Y | Y | | yes |
| F01 | | N | N | 88,778.381 | yes |
| F01 | ST7 | Y | N | | y [symbol] s |
| F01 | TRIML2 | N | Y | 1,641 | |
| F01 | ANKH | N | Y | 1,647 | |
| F01 | ANKH | N | N | 4,220 | |
| F01 | ANKH | N | N | 4,220 | |
| F01 | | N | Y | 4,054.225 | yes |
| F01 | ANKH | N | Y | 3,815 | |
| F01 | | Y | N | | yes |
| F01 | ANKH | N | N | 328 | |
| F01 | ANKH | N | N | 35 | |
| F01 | C5orf51 | N | Y | 23,856.048 | yes |
| F01 | OXCT1 | N | Y | 22,930,878 | yes |
| F01 | HEATR782 | N | Y | 22,182,705 | yes |
| F01 | PLCXD3 | N | N | 12,991.244 | yes |
| F01 | OXCT1 | N | Y | 13,4 [symbol] 7,4 [symbol] 0 | yes |
| F01 | OXCT1 | N | N | 13,182,348 | yes |
| F01 | | N | N | 675,443 | yes |
| F01 | OXCT1 | N | Y | 492.52 [symbol] | |
| F01 | OXCT1 | N | N | 57,77 [symbol] | |
| F01 | CDYL | N | Y | 617 | |
| F01 | SDK1 | N | Y | 742 | |
| F01 | RELN | N | Y | 2,002 | |
| F01 | | Y | Y | | yes |
| F01 | PGCP | N | Y | 395 | |
| F01 | VPS13A | N | N | 4,398 | |
| F01 | VP [symbol] 13A | N | N | 4,397 | |
| F01 | LPAR1 | N | Y | 1,701 | |
| F01 | LPAR1 | N | Y | 1,711 | |
| F01 | NACC2 | N | Y | 3, [symbol] 45 | |
| F01 | NARG2 | Y | N | | yes |
| F01 | NARG2 | Y | N | | yes |
| F01 | BMPR1A | N | Y | 543 | |
| F01 | ATM | N | Y | 95,627 | |
| F01 | BICD1 | N | Y | 5,201 | |
| F01 | | N | Y | 61,511,597 | yes |
| F01 | FAM155A | N | N | 55,216,47 [symbol] | yes |
| F01 | | N | Y | 18,558,799 | yes |
| F01 | GRP175 | N | Y | 1,77 [symbol] | |
| F01 | RABEP1 | N | Y | 493 | |
| F01 | ACCN1 | N | Y | 3,310 | |
| F01 | WNT3 | N | Y | 332 | |
| F01 | SKAP1 | N | Y | 2,153 | |
| F01 | | N | Y | 1,167 | |
| F01 | TMIG [symbol] 2 | N | Y | 503 | |
| F01 | ZNF702P | N | Y | 551 | |
| F01 | REP [symbol] 2 | N | Y | 2,272 | |
| F01 | SH3KBP1 | N | N | 494 | |
| F01 | SH3KBP1 | N | N | 549 | |

[symbol]

indicates data missing or illegible when filed

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the medical sciences are intended to be within the scope of the following claims.

1. A method for detecting NOTCH2 variants associated with splenic marginal zone lymphoma (SMZL) in a subject, comprising:
a) contacting a sample from a subject with a NOTCH2 variant detection assay under conditions that the presence of a NOTCH2 variant associated with SMZL is determined; and
b) diagnosing said subject with SMZL when said NOTCH2 variants are present in said sample.

2. The method of claim 1, wherein said NOTCH2 variant encodes a loss of function mutation.

3. The method of claim 2, wherein said loss of function mutation is a truncation mutation.

4. The method of claim 3, wherein said truncation results in a non-functional PEST domain of said NOTCH2 polypeptide.

5. The method of claim 2, wherein said mutation is one or more mutations selected from the group consisting of c.6909dupC (p.I2304fsX9), c.7198C>T (p.R2400X), c.4999G>A (p.V 1667I), c.6304A>T (p.K2102X), c.6824C>A (p.A2275D), c.6834delinsGCACG (p.T2280fsX12), c.6853C>T (p.Q2285X), c.6868G>A (p.E2290X), c.6873delG (p.K2292fsX3), c.6909delC (p.I2304fsX2), c.6909delC (p.I2304fsX2) plus c.7072A>G (p.M2358V), c.6909dupC (p.I2304fsX9), c.6910delinsCCC (p.I2304fsX3), c.6973C>T (p.Q2325X), and c.7231G>T (p.E2411X).

6. The method of claim 1, wherein said determining comprises detecting variant NOTCH2 nucleic acids or polypeptides.

7. The method of claim 1, wherein said detecting variant NOTCH2 nucleic acids comprises one or more nucleic acid detection method selected from the group consisting of sequencing, amplification and hybridization.

8. The method of claim 1, wherein said biological sample is selected from the group consisting of a tissue sample, a cell sample, and a blood sample.

9. The method of claim 1, wherein said determining comprises a computer implemented method.

10. The method of claim 8, wherein said computer implemented method comprises analyzing NOTCH2 variant information and displaying said information to a user.

11. The method of claim 1, further comprising the step of treating said subject for SMZL and monitoring said subject for the presence of NOTCH2 variants associated with SMZL.

12. The method of claim 1, further comprising the step of treating said subject for SMZL under condition such that at least one symptom of said SMZL is diminished or eliminated.

13. The method of claim 1, further comprising the step of detecting a variant in one or more additional genes.

14. The method of claim 13, wherein said one or more genes are selected from the group consisting of those described in Tables 5 and 6.

15. Use of a variant NOTCH2 nucleic acid or polypeptide for detecting SMZL in a subject.

16. The use of claim 15, wherein said NOTCH2 variant encodes a loss of function mutation.

17. The use of claim 16, wherein said loss of function mutation is a truncation mutation.

18. The use of claim 17, wherein said truncation results in a non-functional PEST domain of said NOTCH2 polypeptide.

19. The use of claim 15, wherein said mutation is one or more mutations selected from the group consisting of c.6909dupC (p.I2304fsX9), c.7198C>T (p.R2400X), c.4999G>A (p.V 1667I), c.6304A>T (p.K2102X), c.6824C>A (p.A2275D), c.6834delinsGCACG (p.T2280fsX12), c.6853C>T (p.Q2285X), c.6868G>A (p.E2290X), c.6873delG (p.K2292fsX3), c.6909delC (p.I2304fsX2), c.6909delC (p.I2304fsX2) plus c.7072A>G (p.M2358V), c.6909dupC (p.I2304fsX9), c.6910delinsCCC (p.I2304fsX3), c.6973C>T (p.Q2325X), and c.7231G>T (p.E2411X).

20. A method of determining a decreased time to adverse outcome in a subject diagnosed with SMZL, comprising:
a) contacting a sample from a subject with a NOTCH2 variant detection assay under conditions that the presence of a NOTCH2 variant associated with SMZL is determined; and
c) detecting a decreased time to adverse outcome in said subject when said NOTCH2 variants are present in said sample.

21. The method of claim 20, wherein said adverse outcome is selected from the group consisting of relapse of SMZL, metastasis, or death.

22. The method of claim 20, wherein said NOTCH2 variant encodes a loss of function mutation.

23. The method of claim 21, wherein said loss of function mutation is a truncation mutation.

24. The method of claim 22, wherein said truncation results in a non-functional PEST domain of said NOTCH2 polypeptide.

25. The method of claim 21, wherein said mutation is one or more mutations selected from the group consisting of c.6909dupC (p.I2304fsX9), c.7198C>T (p.R2400X), c.4999G>A (p.V1667I), c.6304A>T (p.K2102X), c.6824C>A (p.A2275D), c.6834delinsGCACG (p.T2280fsX12), c.6853C>T (p.Q2285X), c.6868G>A (p.E2290X), c.6873delG (p.K2292fsX3), c.6909delC (p.I2304fsX2), c.6909delC (p.I2304fsX2) plus c.7072A>G (p.M2358V), c.6909dupC (p.I2304fsX9), c.6910delinsCCC (p.I2304fsX3), c.6973C>T (p.Q2325X), and c.7231G>T (p.E2411X).

26. The method of claim 20, further comprising the step of detecting a variant in one or more additional genes.

27. The method of claim 26, wherein said one or more genes are selected from the group consisting of those described in Tables 5 and 6.


SEQUENCE LISTING (248 sequences)

SEQ ID NO: 1 - DNA, 23 bp, Artificial Sequence
  Synthetic
gtggaggttttctagaaacctca

SEQ ID NO: 2 - DNA, 21 bp, Artificial Sequence
  Synthetic
gcacaatactggctcagacag

SEQ ID NO: 3 - DNA, 19 bp, Artificial Sequence
  Synthetic
gagtcaggctgtgccagta

SEQ ID NO: 4 - DNA, 20 bp, Artificial Sequence
  Synthetic
ctgttgcaggcctcatcaca

SEQ ID NO: 5 - DNA, 20 bp, Artificial Sequence
  Synthetic
ggtagccgctgtgaactcta

SEQ ID NO: 6 - DNA, 24 bp, Artificial Sequence
  Synthetic
cgagaaactgaagtgtgttagtga

SEQ ID NO: 7 - DNA, 23 bp, Artificial Sequence
  Synthetic
agctccagtctaatctgagctct

SEQ ID NO: 8 - DNA, 20 bp, Artificial Sequence
  Synthetic
caggtggcatcaataccaca

SEQ ID NO: 9 - DNA, 20 bp, Artificial Sequence
  Synthetic
ggtgcaacagtgaggagtgt

SEQ ID NO: 10 - DNA, 22 bp, Artificial Sequence
  Synthetic
tagccttgaagttcagaaacca

SEQ ID NO: 11 - DNA, 21 bp, Artificial Sequence
  Synthetic
atgacatgttctgcctgacct

SEQ ID NO: 12 - DNA, 20 bp, Artificial Sequence
  Synthetic
cctttacaccagtgccactc

SEQ ID NO: 13 - DNA, 23 bp, Artificial Sequence
  Synthetic
atctaatgctgacattgagaggt

SEQ ID NO: 14 - DNA, 21 bp, Artificial Sequence
  Synthetic
agagagagccatgcttacgct

SEQ ID NO: 15 - DNA, 22 bp, Artificial Sequence
  Synthetic
gttgctgttgtcatcattctgt

SEQ ID NO: 16 - DNA, 23 bp, Artificial Sequence
  Synthetic
aatcatgattcaacaagatatgc

SEQ ID NO: 17 - DNA, 21 bp, Artificial Sequence
  Synthetic
gtgtcatggtggaaagtgttg

SEQ ID NO: 18 - DNA, 22 bp, Artificial Sequence
  Synthetic
cagataatggctgacaatggtg

SEQ ID NO: 19 - DNA, 28 bp, Artificial Sequence
  Synthetic
aaacaatgggagataagcagcggtggtg

SEQ ID NO: 20 - DNA, 20 bp, Artificial Sequence
  Synthetic
gacaacaatgtggaaccatg

SEQ ID NO: 21 - DNA, 24 bp, Artificial Sequence
  Synthetic
caaatagagctgtttcaaccatag

SEQ ID NO: 22 - DNA, 21 bp, Artificial Sequence
  Synthetic
attggcatctgcacctgcatc

SEQ ID NO: 23 - DNA, 22 bp, Artificial Sequence
  Synthetic
agatgcagaggactcttctgct

SEQ ID NO: 24 - DNA, 27 bp, Artificial Sequence
  Synthetic
tattattcaagtgactcttctcatgtt

SEQ ID NO: 25 - DNA, 24 bp, Artificial Sequence
  Synthetic
ctacactgtagcctcagctctgat

SEQ ID NO: 26 - DNA, 20 bp, Artificial Sequence
  Synthetic
ccaaatccctgcctttcatc

SEQ ID NO: 27 - DNA, 23 bp, Artificial Sequence
  Synthetic
cattgtgcaagtcatagtgtctt

SEQ ID NO: 28 - DNA, 22 bp, Artificial Sequence
  Synthetic
gaatgggcttataactgaggca

SEQ ID NO: 29 - DNA, 26 bp, Artificial Sequence
  Synthetic
ctcaagagtgttattaacatgtgttc

SEQ ID NO: 30 - DNA, 22 bp, Artificial Sequence
  Synthetic
cttcaggctgaggaaagatctg

SEQ ID NO: 31 - DNA, 20 bp, Artificial Sequence
  Synthetic
tcgcatgcaccatgacattg

SEQ ID NO: 32 - DNA, 25 bp, Artificial Sequence
  Synthetic
ggataaagttactgaactctcagac

SEQ ID NO: 33 - DNA, 21 bp, Artificial Sequence
  Synthetic
ttgccaaggaggcaaaggatg

SEQ ID NO: 34 - DNA, 20 bp, Artificial Sequence
  Synthetic
ctcactgagggaagcacagt

SEQ ID NO: 35 - DNA, 20 bp, Artificial Sequence
  Synthetic
tgggatcttacaggcctcac

SEQ ID NO: 36 - DNA, 20 bp, Artificial Sequence
  Synthetic
ccaggaccataccaaacatc

SEQ ID NO: 37 - DNA, 19 bp, Artificial Sequence
  Synthetic
cgcatggaggtgaatgaga

SEQ ID NO: 38 - DNA, 22 bp, Artificial Sequence
  Synthetic
ccatttctggaatctggtacat

SEQ ID NO: 39 - DNA, 21 bp, Artificial Sequence
  Synthetic
ctaaaggcagtattgcccaac

SEQ ID NO: 40 - DNA, 20 bp, Artificial Sequence
  Synthetic
ctggaggtgaccactgtgac

SEQ ID NO: 41 - DNA, 19 bp, Artificial Sequence
  Synthetic
ggcaggtagctcagaccat

SEQ ID NO: 42 - DNA, 19 bp, Artificial Sequence
  Synthetic
gtcaggagactctggggat

SEQ ID NO: 43 - DNA, 18 bp, Artificial Sequence
  Synthetic
gctgagcgaacacccagt

SEQ ID NO: 44 - DNA, 21 bp, Artificial Sequence
  Synthetic
tgttcctcagcagcatttaca

SEQ ID NO: 45 - DNA, 23 bp, Artificial Sequence
  Synthetic
aggttttctagaaacctcaaact

SEQ ID NO: 46 - DNA, 21 bp, Artificial Sequence
  Synthetic
aatactggctcagacaggtgg

SEQ ID NO: 47 - DNA, 19 bp, Artificial Sequence
  Synthetic
caggctgtgccagtagccc

SEQ ID NO: 48 - DNA, 20 bp, Artificial Sequence
  Synthetic
tgcaggcctcatcacagacg

SEQ ID NO: 49 - DNA, 20 bp, Artificial Sequence
  Synthetic
gccgctgtgaactctacacg

SEQ ID NO: 50 - DNA, 24 bp, Artificial Sequence
  Synthetic
aaactgaagtgtgttagtgacagt

SEQ ID NO: 51 - DNA, 23 bp, Artificial Sequence
  Synthetic
ccagtctaatctgagctcttttg

SEQ ID NO: 52 - DNA, 20 bp, Artificial Sequence
  Synthetic
tggcatcaataccacaataa

SEQ ID NO: 53 - DNA, 20 bp, Artificial Sequence
  Synthetic
caacagtgaggagtgtggtt

SEQ ID NO: 54 - DNA, 22 bp, Artificial Sequence
  Synthetic
cttgaagttcagaaaccaaaca

SEQ ID NO: 55 - DNA, 21 bp, Artificial Sequence
  Synthetic
catgttctgcctgacctgcac

SEQ ID NO: 56 - DNA, 20 bp, Artificial Sequence
  Synthetic
cctttacaccagtgccactc

SEQ ID NO: 57 - DNA, 23 bp, Artificial Sequence
  Synthetic
aatgctgacattgagaggttaat

SEQ ID NO: 58 - DNA, 21 bp, Artificial Sequence
  Synthetic
agagccatgcttacgctttcg

SEQ ID NO: 59 - DNA, 22 bp, Artificial Sequence
  Synthetic
ctgttgtcatcattctgtttat

SEQ ID NO: 60 - DNA, 23 bp, Artificial Sequence
  Synthetic
atgattcaacaagatatgctttt

SEQ ID NO: 61 - DNA, 21 bp, Artificial Sequence
  Synthetic
catggtggaaagtgttgaaaa

SEQ ID NO: 62 - DNA, 22 bp, Artificial Sequence
  Synthetic
taatggctgacaatggtggttc

SEQ ID NO: 63 - DNA, 31 bp, Artificial Sequence
  Synthetic
aatgggagataagcagcggtggtggaggctc

SEQ ID NO: 64 - DNA, 20 bp, Artificial Sequence
  Synthetic
acaatgtggaaccatgggca

SEQ ID NO: 65 - DNA, 24 bp, Artificial Sequence
  Synthetic
tagagctgtttcaaccatagggtt

SEQ ID NO: 66 - DNA, 21 bp, Artificial Sequence
  Synthetic
gcatctgcacctgcatccagg

SEQ ID NO: 67 - DNA, 22 bp, Artificial Sequence
  Synthetic
gcagaggactcttctgctaaca

SEQ ID NO: 68 - DNA, 30 bp, Artificial Sequence
  Synthetic
attcaagtgactcttctcatgttctttacc

SEQ ID NO: 69 - DNA, 24 bp, Artificial Sequence
  Synthetic
actgtagcctcagctctgatgccc

SEQ ID NO: 70 - DNA, 20 bp, Artificial Sequence
  Synthetic
atccctgcctttcatcccta

SEQ ID NO: 71 - DNA, 23 bp, Artificial Sequence
  Synthetic
gtgcaagtcatagtgtcttatac

SEQ ID NO: 72 - DNA, 22 bp, Artificial Sequence
  Synthetic
gggcttataactgaggcactgc

SEQ ID NO: 73 - DNA, 26 bp, Artificial Sequence
  Synthetic
agagtgttattaacatgtgttctgtg

SEQ ID NO: 74 - DNA, 22 bp, Artificial Sequence
  Synthetic
aggctgaggaaagatctgttgg

SEQ ID NO: 75 - DNA, 20 bp, Artificial Sequence
  Synthetic
atgcaccatgacattgtgcg

SEQ ID NO: 76 - DNA, 25 bp, Artificial Sequence
  Synthetic
aaagttactgaactctcagacagtt

SEQ ID NO: 77 - DNA, 21 bp, Artificial Sequence
  Synthetic
caaggaggcaaaggatgccaa

SEQ ID NO: 78 - DNA, 20 bp, Artificial Sequence
  Synthetic
ctgagggaagcacagtgctg

SEQ ID NO: 79 - DNA, 20 bp, Artificial Sequence
  Synthetic
atcttacaggcctcacccaa

SEQ ID NO: 80 - DNA, 20 bp, Artificial Sequence
  Synthetic
gaccataccaaacatctcat

SEQ ID NO: 81 - DNA, 19 bp, Artificial Sequence
  Synthetic
atggaggtgaatgagaccc

SEQ ID NO: 82 - DNA, 22 bp, Artificial Sequence
  Synthetic
ccatttctggaatctggtacat

SEQ ID NO: 83 - DNA, 21 bp, Artificial Sequence
  Synthetic
aggcagtattgcccaaccagc

SEQ ID NO: 84 - DNA, 20 bp, Artificial Sequence
  Synthetic
aggtgaccactgtgactggg

SEQ ID NO: 85 - DNA, 19 bp, Artificial Sequence
  Synthetic
ggtagctcagaccattctc

SEQ ID NO: 86 - DNA, 19 bp, Artificial Sequence
  Synthetic
ggagactctggggatggtg

SEQ ID NO: 87 - DNA, 18 bp, Artificial Sequence
  Synthetic
agcgaacacccagtcaca

SEQ ID NO: 88 - DNA, 21 bp, Artificial Sequence
  Synthetic
cctcagcagcatttacaaaag

SEQ ID NO: 89 - DNA, 75 bp, Artificial Sequence
  Synthetic
gatgctcaccctggaggtgaccactgtgactgggtgttcgctcagcagcatttgaggaagcataactgtgctgtg

SEQ ID NO: 90 - DNA, 33 bp, Artificial Sequence
  Synthetic
gatgctcaccctggaggtgaccatgggtgttca

SEQ ID NO: 91 - DNA, 34 bp, Artificial Sequence
  Synthetic
gatgctcaccctggaggtgaccactgggtgttca

SEQ ID NO: 92 - DNA, 33 bp, Artificial Sequence
  Synthetic
atgctcaccctggaggtgaccactgggtgttcg

SEQ ID NO: 93 - DNA, 35 bp, Artificial Sequence
  Synthetic
gctcagcccggaagtgaccactgtgggtgttcgct

SEQ ID NO: 94 - DNA, 35 bp, Artificial Sequence
  Synthetic
gctcaccctggaggtgaccactgtgggtgttcgct

SEQ ID NO: 95 - DNA, 33 bp, Artificial Sequence
  Synthetic
ctcaccctagaccactgtgactgggtgttcgct

SEQ ID NO: 96 - DNA, 33 bp, Artificial Sequence
  Synthetic
tcaccctggaggtgaccactgtgtgttcgctca

SEQ ID NO: 97 - DNA, 33 bp, Artificial Sequence
  Synthetic
tcaccctggaggtgaccactgtgtgttcactca

SEQ ID NO: 98 - DNA, 33 bp, Artificial Sequence
  Synthetic
accctggaggtgaccactgtgactgttcactca

SEQ ID NO: 99 - DNA, 33 bp, Artificial Sequence
  Synthetic
accctggaggtgaccactgtgacgttcactcag

SEQ ID NO: 100 - DNA, 33 bp, Artificial Sequence
  Synthetic
accctggaggctgtgactgggtgttcgctctgc

SEQ ID NO: 101 - DNA, 33 bp, Artificial Sequence
  Synthetic
accctggaggctgtgactgggtgttcactcagc

SEQ ID NO: 102 - DNA, 33 bp, Artificial Sequence
  Synthetic
accctggaggtgaccactgtgactcactaggaa

SEQ ID NO: 103 - DNA, 33 bp, Artificial Sequence
  Synthetic
ccctggaggtctgtgactgggtgttcgctcagc

SEQ ID NO: 104 - DNA, 33 bp, Artificial Sequence
  Synthetic
ccctggaggtgaccactgtgacttcgctcagca

SEQ ID NO: 105 - DNA, 34 bp, Artificial Sequence
  Synthetic
ccctggaggttgtgactgggtgttcactcagcag

SEQ ID NO: 106 - DNA, 33 bp, Artificial Sequence
  Synthetic
cctggaggtgacccggtggctggcgctcagcag

SEQ ID NO: 107 - DNA, 33 bp, Artificial Sequence
  Synthetic
aggtgaccactgggtgttcgctcagcagcattt

SEQ ID NO: 108 - DNA, 33 bp, Artificial Sequence
  Synthetic
tgaccactgtgactgggtgttcgcaacttttga

SEQ ID NO: 109 - DNA, 33 bp, Artificial Sequence
  Synthetic
tgaccactgtgactgggtgtttgcagcatttga

SEQ ID NO: 110 - DNA, 33 bp, Artificial Sequence
  Synthetic
gaccattatgacagggtgttcacagcatttgag

SEQ ID NO: 111 - DNA, 33 bp, Artificial Sequence
  Synthetic
accactgtgagttcactcagcagcatttgagga

SEQ ID NO: 112 - DNA, 34 bp, Artificial Sequence
  Synthetic
accactgtgactgggtgttagcttatttgaggaa

SEQ ID NO: 113 - DNA, 32 bp, Artificial Sequence
  Synthetic
accactgtgactgtgttcactaatttgaggaa

SEQ ID NO: 114 - DNA, 33 bp, Artificial Sequence
  Synthetic
ccactgtgactgggtgttcgctgcagttgagga

SEQ ID NO: 115 - DNA, 33 bp, Artificial Sequence
  Synthetic
ccactgtgactgggtgttcggtgcatttgagga

SEQ ID NO: 116 - DNA, 33 bp, Artificial Sequence
  Synthetic
ccactgtgacgttcactcagcagcatttgagga

SEQ ID NO: 117 - DNA, 33 bp, Artificial Sequence
  Synthetic
ccactgtgactgggtgttcgctaatttgaggaa

SEQ ID NO: 118 - DNA, 33 bp, Artificial Sequence
  Synthetic
ccactttgactgggtgttcacccatttgaggaa

SEQ ID NO: 119 - DNA, 33 bp, Artificial Sequence
  Synthetic
cactgtgactttcgctcagcagcatttgaggaa

SEQ ID NO: 120 - DNA, 33 bp, Artificial Sequence
  Synthetic
gactgggtgttcgctcagcagcagaagcataac

SEQ ID NO: 121 - DNA, 33 bp, Artificial Sequence
  Synthetic
ctgggtgttcgctcagcagcattagcataactg

SEQ ID NO: 122 - DNA, 33 bp, Artificial Sequence
  Synthetic
tgggtgttcacagcacttgaggaagcataactg

SEQ ID NO: 123 - DNA, 33 bp, Artificial Sequence
  Synthetic
tgggtgttcgcagcatttgagaagcataactgt

SEQ ID NO: 124 - DNA, 33 bp, Artificial Sequence
  Synthetic
tgggtgtgcgcagcatttgagaagcataactgt

SEQ ID NO: 125 - DNA, 32 bp, Artificial Sequence
  Synthetic
gggtgttcgctcagcagcatttgcataactgt

SEQ ID NO: 126 - DNA, 33 bp, Artificial Sequence
  Synthetic
tgttcactcagcagcatttgaggtaactgtgct

SEQ ID NO: 127 - DNA, 33 bp, Artificial Sequence
  Synthetic
ttcgctcagcagcatttgaggaactgtgctgtg

SEQ ID NO: 128 - DNA, 25 bp, Artificial Sequence
  Synthetic
aaatgctgctgagygaacacccagt

SEQ ID NO: 129 - DNA, 25 bp, Artificial Sequence
  Synthetic
aaatgctgctgagcgaacacccagt

SEQ ID NO: 130 - DNA, 73 bp, Artificial Sequence
  Synthetic
tactgcctttagggatgagctggaaagtcacaatggggggcaagggctcccgaggggtggttatgtgcttccc

SEQ ID NO: 131 - DNA, 32 bp, Artificial Sequence
  Synthetic
ctgcctttaggggagctggaaaatggggggca

SEQ ID NO: 132 - DNA, 34 bp, Artificial Sequence
  Synthetic
ctgcctttagggatgagctggaaaatggggggca

SEQ ID NO: 133 - DNA, 33 bp, Artificial Sequence
  Synthetic
tgcctttagggatgagctggaaaatggggggca

SEQ ID NO: 134 - DNA, 32 bp, Artificial Sequence
  Synthetic
gcctttagggatagctggaaagatgggggggc

SEQ ID NO: 135 - DNA, 33 bp, Artificial Sequence
  Synthetic
gcctttagggatgagctggaaagtgggggggca

SEQ ID NO: 136 - DNA, 33 bp, Artificial Sequence
  Synthetic
gcctttagggatgagctggaaagtggggggcaa

SEQ ID NO: 137 - DNA, 32 bp, Artificial Sequence
  Synthetic
gcctttagggatagctggaaagtggggggcaa

SEQ ID NO: 138 - DNA, 33 bp, Artificial Sequence
  Synthetic
cctttagggagggaaagtcacaatggggggcaa

SEQ ID NO: 139 - DNA, 33 bp, Artificial Sequence
  Synthetic
cctttagggatggaaagtcacaatggggggcaa

SEQ ID NO: 140 - DNA, 34 bp, Artificial Sequence
  Synthetic
cctttagggatgagctggaaagtcggggggcaag

SEQ ID NO: 141 - DNA, 33 bp, Artificial Sequence
  Synthetic
ctttagggatgagctggaaagtcggggggcaag

SEQ ID NO: 142 - DNA, 33 bp, Artificial Sequence
  Synthetic
ctttagggatgagctggaaagtcgggggcaagg

SEQ ID NO: 143 - DNA, 33 bp, Artificial Sequence
  Synthetic
ttagggatgaactggaaagtcacgggcaagggc

SEQ ID NO: 144 - DNA, 33 bp, Artificial Sequence
  Synthetic
tagggatgagctggaaagtcacagtcaagggct

SEQ ID NO: 145 - DNA, 33 bp, Artificial Sequence
  Synthetic
tagggatgagctggaaagtcacaggcaagggct

SEQ ID NO: 146 - DNA, 33 bp, Artificial Sequence
  Synthetic
tagggatgaggtcacaatgggggggcaagggct

SEQ ID NO: 147 - DNA, 33 bp, Artificial Sequence
  Synthetic
agggatgagctggaaagtcacaagggcaagggc

SEQ ID NO: 148 - DNA, 33 bp, Artificial Sequence
  Synthetic
agggatgagctggaaagtcacaagcaagggctc

SEQ ID NO: 149 - DNA, 33 bp, Artificial Sequence
  Synthetic
gggatgagctggaaagtcacaatcaagggctcc

SEQ ID NO: 150 - DNA, 33 bp, Artificial Sequence
  Synthetic
gggatgagcttcacaatggggggcaagggctcc

SEQ ID NO: 151 - DNA, 33 bp, Artificial Sequence
  Synthetic
gggatgagctcacaatggggggcaagggctccc

SEQ ID NO: 152 - DNA, 33 bp, Artificial Sequence
  Synthetic
ggatgagctggaaagtcacaatgaatggctccc

SEQ ID NO: 153 - DNA, 33 bp, Artificial Sequence
  Synthetic
gatgagctggaaagtcacaatggaagggctccc

SEQ ID NO: 154 - DNA, 33 bp, Artificial Sequence
  Synthetic
gatgagctggaaagtcacaatggagggctcccg

SEQ ID NO: 155 - DNA, 32 bp, Artificial Sequence
  Synthetic
atgagctggaaatcacaatgggcaagggctcc

SEQ ID NO: 156 - DNA, 33 bp, Artificial Sequence
  Synthetic
atgagctggaaagtcacaatgggaagggctccc

SEQ ID NO: 157 - DNA, 33 bp, Artificial Sequence
  Synthetic
atgagctggaaagtcacaatgggatgggctccc

SEQ ID NO: 158 - DNA, 33 bp, Artificial Sequence
  Synthetic
gagctggaaagtcacaatggggggctcccgagg

SEQ ID NO: 159 - DNA, 33 bp, Artificial Sequence
  Synthetic
agctggaaagtcacaatggggggctcccgaggg

SEQ ID NO: 160 - DNA, 33 bp, Artificial Sequence
  Synthetic
agctggaaagtcacaatggggggcttctgaggg

SEQ ID NO: 161 - DNA, 33 bp, Artificial Sequence
  Synthetic
ctggaaagccacaatggggggcacccgaggggt

SEQ ID NO: 162 - DNA, 33 bp, Artificial Sequence
  Synthetic
tggaaagtcacaatggggggcaaccgaggggtg

SEQ ID NO: 163 - DNA, 34 bp, Artificial Sequence
  Synthetic
tggaaggggacaatggggggcaagccgaggggtg

SEQ ID NO: 164 - DNA, 33 bp, Artificial Sequence
  Synthetic
tggaaagtcacaaggggtgcaagccgaggggtg

SEQ ID NO: 165 - DNA, 33 bp, Artificial Sequence
  Synthetic
tggaaagtcacaaggggggcaagccgaggggtg

SEQ ID NO: 166 - DNA, 34 bp, Artificial Sequence
  Synthetic
tggaaagtcacaatggggggcaagccgaggggtg

SEQ ID NO: 167 - DNA, 34 bp, Artificial Sequence
  Synthetic
tggaaagtcacaatggggggcaagcgaggggtgg

SEQ ID NO: 168 - DNA, 33 bp, Artificial Sequence
  Synthetic
gaaagtcacaatggggggcaagggaggggtggt

SEQ ID NO: 169 - DNA, 34 bp, Artificial Sequence
  Synthetic
gaaagacagaatggggggcaagggaggggtggtt

SEQ ID NO: 170 - DNA, 34 bp, Artificial Sequence
  Synthetic
gaaagtcacaatggggggcaagggggggtggtta

SEQ ID NO: 171 - DNA, 33 bp, Artificial Sequence
  Synthetic
aaagtcacaatggggggcaagggaggggtggtt

SEQ ID NO: 172 - DNA, 33 bp, Artificial Sequence
  Synthetic
aaagtcacaagcaagggctcccgaggggtggtt

SEQ ID NO: 173 - DNA, 33 bp, Artificial Sequence
  Synthetic
aaagtcacaatggggggcaagggcggggtggtt

SEQ ID NO: 174 - DNA, 34 bp, Artificial Sequence
  Synthetic
aaagtcactagcaagggctcccggggggtggtta

SEQ ID NO: 175 - DNA, 33 bp, Artificial Sequence
  Synthetic
aagtcaggatggggggcaagggcaggggtggtt

SEQ ID NO: 176 - DNA, 33 bp, Artificial Sequence
  Synthetic
aagtcacaatggggggcaagggcggggtggtta

SEQ ID NO: 177 - DNA, 33 bp, Artificial Sequence
  Synthetic
aagtcacaatggggggcaagggctgggtggtta

SEQ ID NO: 178 - DNA, 33 bp, Artificial Sequence
  Synthetic
aagtcacgatggggggcaagggcggggtggtta

SEQ ID NO: 179 - DNA, 33 bp, Artificial Sequence
  Synthetic
aagtcaggatggggggcaagggcggggtggtta

SEQ ID NO: 180 - DNA, 32 bp, Artificial Sequence
  Synthetic
aagtcacaatggggggcaagggggggtggtta

SEQ ID NO: 181 - DNA, 35 bp, Artificial Sequence
  Synthetic
aagtcacaatgggggggcaagggctggtggttatg

SEQ ID NO: 182 - DNA, 33 bp, Artificial Sequence
  Synthetic
agtcagaatggggggcaagggctaggggtggtt

SEQ ID NO: 183 - DNA, 33 bp, Artificial Sequence
  Synthetic
gtcacaatggggggcaagggctcgggtggttat

SEQ ID NO: 184 - DNA, 33 bp, Artificial Sequence
  Synthetic
gtcacaaaggggggcaagggctcgggtggttat

SEQ ID NO: 185 - DNA, 33 bp, Artificial Sequence
  Synthetic
gtcacaatgggggggcaagggctgggtggttat

SEQ ID NO: 186 - DNA, 33 bp, Artificial Sequence
  Synthetic
gtcacaatggggggcaagggctcggtggttatg

SEQ ID NO: 187 - DNA, 33 bp, Artificial Sequence
  Synthetic
tcacaatggggggcaagggctccgggtggttat

SEQ ID NO: 188 - DNA, 33 bp, Artificial Sequence
  Synthetic
tcacaatggggggcaagggctccggtggttatg

SEQ ID NO: 189 - DNA, 33 bp, Artificial Sequence
  Synthetic
tcacaatggggggctcccgaggggtggttatgt

SEQ ID NO: 190 - DNA, 33 bp, Artificial Sequence
  Synthetic
ccacaatggggggctcccgaggggtggttatgt

SEQ ID NO: 191 - DNA, 32 bp, Artificial Sequence
  Synthetic
cacaatggggggaagggctccgtggttatgtg

SEQ ID NO: 192 - DNA, 33 bp, Artificial Sequence
  Synthetic
acaatggggggcaagggctcccgtggttatgtg

SEQ ID NO: 193 - DNA, 33 bp, Artificial Sequence
  Synthetic
caatgggggggcaagggctcccgtggttatgtg

SEQ ID NO: 194 - DNA, 33 bp, Artificial Sequence
  Synthetic
caatggggggcaagggctcccgagttatgtgct

SEQ ID NO: 195 - DNA, 33 bp, Artificial Sequence
  Synthetic
aatggggggcaagggctcccgagttatgtgctt

SEQ ID NO: 196 - DNA, 32 bp, Artificial Sequence
  Synthetic
aatggggggcaagggctcccgattatgtgctt

SEQ ID NO: 197 - DNA, 35 bp, Artificial Sequence
  Synthetic
aatgggggggcaagggctcccgaggtatgtgcttc

SEQ ID NO: 198 - DNA, 34 bp, Artificial Sequence
  Synthetic
aatggggggcaagggctcccgaggtatgtgcttt

SEQ ID NO: 199 - DNA, 33 bp, Artificial Sequence
  Synthetic
atgggggggcaagggctcccgaggttatgtgct

SEQ ID NO: 200 - DNA, 33 bp, Artificial Sequence
  Synthetic
atgggggagcaagggctcccgagttatgtgctt

SEQ ID NO: 201 - DNA, 33 bp, Artificial Sequence
  Synthetic
atggggggcaagggctcccgaggtatgtgcttc

SEQ ID NO: 202 - DNA, 33 bp, Artificial Sequence
  Synthetic
atggggggcaacggctcccgaggtatgtgcttc

SEQ ID NO: 203 - DNA, 33 bp, Artificial Sequence
  Synthetic
atggggggcaagggctctcgaggtatgtgcttc

SEQ ID NO: 204 - DNA, 33 bp, Artificial Sequence
  Synthetic
atggggtgcaagggctcacgaggtatgtgcttc

SEQ ID NO: 205 - DNA, 21 bp, Artificial Sequence
  Synthetic
cttgccccccmwtkkkrmytt

SEQ ID NO: 206 - DNA, 21 bp, Artificial Sequence
  Synthetic
cttgccccccattgtgacttt

SEQ ID NO: 207 - DNA, 77 bp, Artificial Sequence
  Synthetic
atgctcaccctggaggtgaccactgtgactgggtgttcgctcagcagcatttgaggaagcataactgtgctgtgaag

SEQ ID NO: 208 - DNA, 33 bp, Artificial Sequence; identical to SEQ ID NO: 92
  Synthetic

SEQ ID NO: 209 - DNA, 33 bp, Artificial Sequence
  Synthetic
atgctcaccctggaggtgaccactgggtgttca

SEQ ID NO: 210 - DNA, 33 bp, Artificial Sequence
  Synthetic
tgttcaccctggaggtgaccacttgggtgttcg

SEQ ID NO: 211 - DNA, 32 bp, Artificial Sequence
  Synthetic
tgctcaccctgggtgcccactcggtgttcgct

SEQ ID NO: 212 - DNA, 33 bp, Artificial Sequence
  Synthetic
ctcaccctggaccactgtgactgtgtgttcgct

SEQ ID NO: 213 - DNA, 33 bp, Artificial Sequence
  Synthetic
ttcaccctggccactgtgactgggtgttcgctc

SEQ ID NO: 214 - DNA, 33 bp, Artificial Sequence
  Synthetic
ctcaccctggccactgtgactgggtgttcgctc

SEQ ID NO: 215 - DNA, 32 bp, Artificial Sequence
  Synthetic
ctcaccctggaggtgaccactgttgttcactc

SEQ ID NO: 216 - DNA, 34 bp, Artificial Sequence
  Synthetic
ctcaccctggaggtgaccactgtgtgttcgctca

SEQ ID NO: 217 - DNA, 33 bp, Artificial Sequence
  Synthetic
accctggaggactgtgactgggtgttcactcag

SEQ ID NO: 218 - DNA, 33 bp, Artificial Sequence
  Synthetic
cccacgaggttgtgactgggtgttcgctcagca

SEQ ID NO: 219 - DNA, 33 bp, Artificial Sequence
  Synthetic
ccctggaggtgaccactgtgacttcactcagca

SEQ ID NO: 220 - DNA, 33 bp, Artificial Sequence
  Synthetic
cctggaggtgtgtgactgggtgttcgctcagca

SEQ ID NO: 221 - DNA, 33 bp, Artificial Sequence; identical to SEQ ID NO: 107
  Synthetic

SEQ ID NO: 222 - DNA, 33 bp, Artificial Sequence
  Synthetic
accactgtgactgggtgttcactgcatttgagg

SEQ ID NO: 223 - DNA, 33 bp, Artificial Sequence
  Synthetic
ccactgtgactgggtgttcactcgcatttgagg

SEQ ID NO: 224 - DNA, 33 bp, Artificial Sequence
  Synthetic
ccactgtgacgttcgctcagcagcatttgagga

SEQ ID NO: 225 - DNA, 33 bp, Artificial Sequence
  Synthetic
ccactgtgactgggtgttgggtaatttgaggaa

SEQ ID NO: 226 - DNA, 33 bp, Artificial Sequence
  Synthetic
gtgactgggtgttcactcagcagaggaagcata

SEQ ID NO: 227 - DNA, 33 bp, Artificial Sequence
  Synthetic
tgactgggtgttcgctcagcagcggaagcataa

SEQ ID NO: 228 - DNA, 33 bp, Artificial Sequence
  Synthetic
tgggtgttcactcagcagcatttagcataactg

SEQ ID NO: 229 - DNA, 33 bp, Artificial Sequence
  Synthetic
gggtgttcgctcagcagcatttgcataactgtg

SEQ ID NO: 230 - DNA, 33 bp, Artificial Sequence
  Synthetic
ggtgttcactcagcagcatttgaataactgtgc

SEQ ID NO: 231 - DNA, 33 bp, Artificial Sequence
  Synthetic
tgttcgctcagcagcatttgaggtaactgtgct

SEQ ID NO: 232 - DNA, 33 bp, Artificial Sequence
  Synthetic
tcgctcagcagcatttggggaagctgtgctgtg

SEQ ID NO: 233 - DNA, 33 bp, Artificial Sequence
  Synthetic
actcagcagcaggaagcatactgtgctgtgaag

SEQ ID NO: 234 - DNA, 33 bp, Artificial Sequence
  Synthetic
actcagaagcatttgaggaagcatgctgtgaag

SEQ ID NO: 235 - DNA, 11 bp, Artificial Sequence
  Synthetic
ctgtcrtcagt

SEQ ID NO: 236 - DNA, 11 bp, Artificial Sequence
  Synthetic
gcaagwagtct

SEQ ID NO: 237 - DNA, 11 bp, Artificial Sequence
  Synthetic
tccagmtgagg

SEQ ID NO: 238 - DNA, 14 bp, Artificial Sequence
  Synthetic
ggcacccatcctgg

SEQ ID NO: 239 - DNA, 11 bp, Artificial Sequence
  Synthetic
ctcccyagagc

SEQ ID NO: 240 - DNA, 11 bp, Artificial Sequence
  Synthetic
cacctraaggg

SEQ ID NO: 241 - DNA, 13 bp, Artificial Sequence
  Synthetic
aagggaagcacat

SEQ ID NO: 242 - DNA, 13 bp, Artificial Sequence
  Synthetic
cccccattgtgac

SEQ ID NO: 243 - DNA, 11 bp, Artificial Sequence
  Synthetic
ccatgrtgccc

SEQ ID NO: 244 - DNA, 13 bp, Artificial Sequence
  Synthetic
ccccattgtgact

SEQ ID NO: 245 - DNA, 14 bp, Artificial Sequence
  Synthetic
ccccattgtgactt

SEQ ID NO: 246 - DNA, 11 bp, Artificial Sequence
  Synthetic
agcctyagtcc

SEQ ID NO: 247 - DNA, 11 bp, Artificial Sequence
  Synthetic
ctgagygaaca

SEQ ID NO: 248 - DNA, 11 bp, Artificial Sequence
  Synthetic
agggtkagcat